(19) United States
(12) Patent Application Publication
(10) Pub. No.: US 2026/0274773 A1
(43) Pub. Date: Sep. 17, 2026

(54) COMPOUND, COMPOUND PRODUCTION METHOD, CURABLE RESIN COMPOSITION, AND CURED PRODUCT OF SAME

(71) Applicant: NIPPON KAYAKU KABUSHIKI KAISHA, Tokyo (JP)

(72) Inventors: Takayuki TOHJIMA, Tokyo (JP); Masato SEKI, Tokyo (JP); Hiroki KONDO, Tokyo (JP); Masanori HASHIMOTO, Tokyo (JP)

(73) Assignee: NIPPON KAYAKU KABUSHIKI KAISHA, Tokyo (JP)

(21) Appl. No.: 19/167,503

(22) PCT Filed: Mar. 18, 2024
(86) PCT No.: PCT/JP2024/010524

§ 371 Date: Sep. 22, 2025

(30) Foreign Application Priority Data
Mar. 23, 2023 (JP) 2023-046177
Aug. 28, 2023 (JP) 2023-137647
Aug. 28, 2023 (JP) 2023-137648
Dec. 5, 2023 (JP) 2023-204993
Dec. 20, 2023 (JP) 2023-214747

(51) Int. Cl. C07C 13/567; C08F 12/34

(52) U.S. Cl. CPC C07C 13/567; C08F 12/34

(57) ABSTRACT

The present invention provides: a compound represented by formula (1) and having excellent low dielectric properties; a curable resin composition containing the compound; and a cured product thereof. In formula (1), R₁'s, which exist in a quantity of more than one, each independently represent a hydrogen atom or a hydrocarbon group having 1-5 carbon atoms. R₂'s, which exist in a quantity of more than one, each independently represent a hydrocarbon group represented by formula (a). l's, which exist in a quantity of more than one, each independently represent an integer of 1-4. m's, which exist in a quantity of more than one, each independently represent an integer of 0-4, and the average value mₐᵥₑ of m satisfies 0<mₐᵥₑ≤4. In formula (a), * represents the site of binding to a fluorene structure in formula (1). R₃'s, which exist in a quantity of more than one, each independently represent a hydrogen atom or a hydrocarbon group having 1-5 carbon atoms. E₁ represents a direct bond or a methylene group. E₂ and E₃ each represent a hydrogen atom, a hydrocarbon group having 1-5 carbon atoms, or a substituted or unsubstituted phenyl group. n represents an integer of 1-5.

### TECHNICAL FIELD

The present invention relates to a compound having a specific structure, a method for producing the compound, a curable resin composition and a cured product thereof, which are suitably used for electrical and electronic components such as semiconductor encapsulants, printed wiring boards and build-up laminates, lightweight and high-strength materials such as carbon fiber reinforced plastics and glass fiber reinforced plastics, and 3D printing applications.

### BACKGROUND ART

In recent years, the laminates on which electric and electronic components are mounted have come to be used in a wider range of fields, and the required characteristics have become more extensive and sophisticated. Conventional semiconductor chips were mainly mounted on metal lead frames, but semiconductor chips with high processing power, such as central processing units (hereinafter referred to as CPUs), are increasingly being mounted on laminates made of polymer materials.

The fifth generation communication system “5G”, which is currently being developed at an accelerated pace, is expected to further increase capacity and speed. In 5G, the frequency used will become higher, but reducing transmission loss is important to achieve high-speed communication using high frequencies, and further low dielectric properties will be required for the board material. The transmission loss occurring on the printed circuit board is due to conductor loss and dielectric loss. As described in Non-Patent Document 1, the dielectric loss αD is proportional to the square root of the relative dielectric constant εᵣ of the dielectric and the dielectric loss tangent tan δ, so it can be said that improving the dielectric loss tangent tan δ, which has a higher contribution than the relative dielectric constant εᵣ, is effective in reducing transmission loss. Thermoplastic materials such as PTFE (polytetrafluoroethylene) and LCP (liquid crystal polymer) are examples of materials with low dielectric loss tangent, but they are poor in moldability compared to thermosetting resins.

In light of this, the development of thermosetting resins with excellent low dielectric properties is desired.

In light of this background, polymeric materials with excellent dielectric properties have been studied. For example, Patent Document 1 proposes a thermosetting resin composition containing an imide compound having a maleimide group and a phenol aralkyl resin having an aliphatic unsaturated bond. However, on the other hand, since phenolic hydroxy groups that do not participate in the reaction remain during the curing reaction, the electrical properties are not sufficient. Patent Document 2 also discloses an allyl ether-modified biphenyl aralkyl novolac resin to which an allyl group is added together with a phenolic hydroxy group. However, it has been shown that the allyl ether-modified biphenyl aralkyl novolac resin undergoes Claisen rearrangement at 190° C., and at 200° C., which is the molding temperature of a general substrate, phenolic hydroxy groups that do not contribute to the curing reaction are generated, so the electrical properties are not satisfactory.

### CITATION LIST

### Non-Patent Literature

Non-Patent Literature 1: “Consideration of signal loss factor in the high-speed Signal transmission on a printed Circuit boards,” 29th Spring Conference of the Japan Institute of Electronics Packaging, Session ID: 16P1-17, 2015

### Patent Literature

Patent Literature 1: Japanese Patent Publication No. 04-359911
Patent Literature 2: International Publication No. 2016/002704

### SUMMARY OF INVENTION

### Technical Problem

The present invention has been made in view of the above circumstances, and an object of the present invention is to provide a compound having excellent low dielectric properties, a curable resin composition, and a cured product thereof.

### Solution to Problem

That is, the present invention relates to the following [1] to [7]. In the present invention, “(Numerical value 1) to (Numerical value 2)” indicates that the upper and lower limits are included.
[1] A compound represented by the following formula (1):

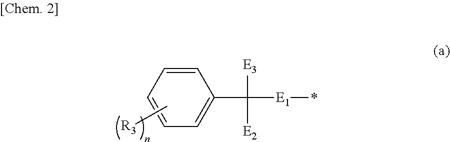

In the above formula (1), each of the multiple R₁ independently represents a hydrogen atom or a hydrocarbon group having 1 to 5 carbon atoms. Each of the multiple R₂ independently represents a hydrocarbon group represented by the following formula (a). Each of the multiple 1 independently represents an integer of 1 to 4. Each of the multiple m independently represents an integer of 0 to 4, with the average mₐᵥₑ of the values m being 0<mₐᵥₑ≤4.

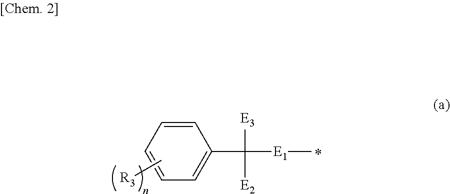

In the above formula (a), * represents a bonding position to the fluorene structure of formula (1). Each of the multiple R₃ independently represents a hydrogen atom or a hydrocarbon group having 1 to 5 carbon atoms. E₁ represents a direct bond or a methylene group. E₂ and E₃ represent a hydrogen atom, a hydrocarbon group having 1 to 5 carbon atoms, or a substituted or unsubstituted phenyl group. n represents an integer of 1 to 5.
[2] The compound according to the above item [1], wherein the hydrocarbon group represented by formula (a) is one or more selected from the following formulas (a-1) to (a-6):

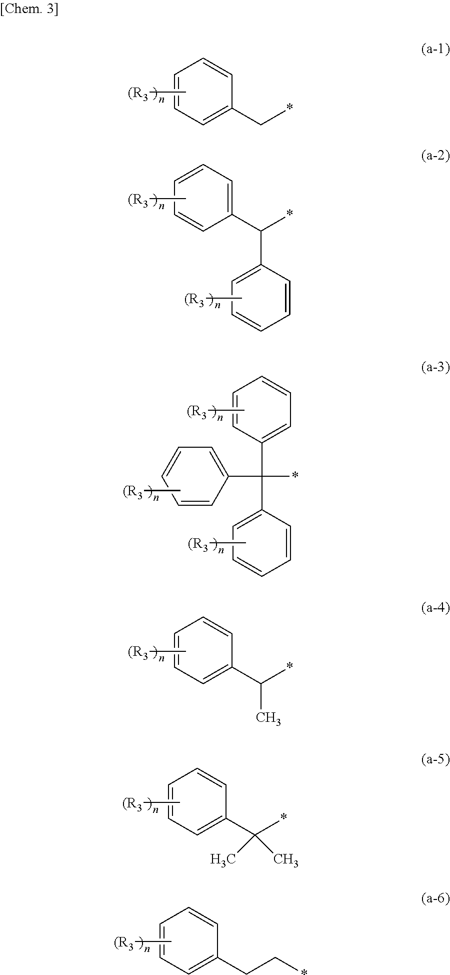

In the above formulas (a-1) to (a-6), * represents a bonding position to the fluorene structure of formula (1). Each of the multiple R₃ independently represents a hydrogen atom or a hydrocarbon group having 1 to 5 carbon atoms. Each of the multiple n independently represents an integer of 1 to 5.
[3] A compound obtained by reacting a compound represented by the following formula (A) with a compound represented by the following formula (B).

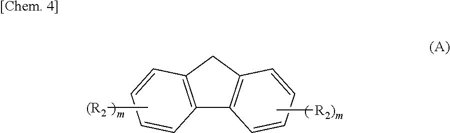

In the above formula (A), each of the multiple R₂ represents a hydrocarbon group represented by the following formula (a). Each of the multiple m independently represents an integer of 0 to 4, and the average mₐᵥₑ of the values m is 0<mₐᵥₑ≤4.

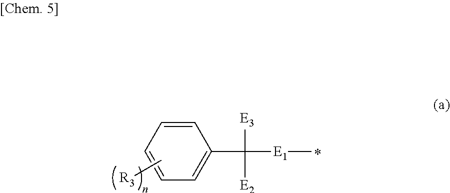

In the above formula (a), * represents a bonding position to the fluorene structure of formula (A). Each of the multiple R₃ independently represents a hydrogen atom or a hydrocarbon group having 1 to 5 carbon atoms. E₁ represents a direct bond or a methylene group. E₂ and E₃ represent a hydrogen atom, a hydrocarbon group having 1 to 5 carbon atoms, or a substituted or unsubstituted phenyl group. n represents an integer of 1 to 5.

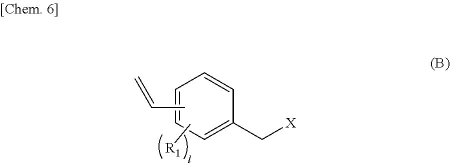

In the formula (B), each of the multiple R₁ independently represents a hydrogen atom or a hydrocarbon group having 1 to 5 carbon atoms. Each of the multiple 1 independently represents an integer of 1 to 4. X represents a halogen atom.
[4] A curable resin composition containing the compound according to any one of the preceding items [1] to [3].
[5] A cured product obtained by curing the compound according to any one of the preceding items [1] to [3].
[6] A cured product obtained by curing the curable resin composition according to the preceding item [4].
[7] A method for producing a compound obtained by reacting a compound represented by the following formula (A) with a compound represented by the following formula (B) in an aprotic polar solvent in the presence of a basic catalyst.

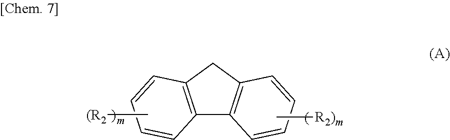

In the above formula (A), each of the multiple R₂ independently represents a hydrocarbon group represented by the following formula (a). Each of the multiple m independently represents an integer of 0 to 4, and the average mₐᵥₑ of the values m is 0<mₐᵥₑ≤4.

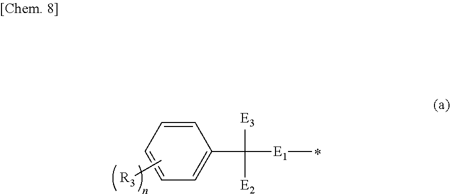

In the above formula (a), * represents a bonding position to the fluorene structure of formula (A). Each of the multiple R₃ independently represents a hydrogen atom or a hydrocarbon group having 1 to 5 carbon atoms. E₁ represents a direct bond or a methylene group. E₂ and E₃ represent a hydrogen atom, a hydrocarbon group having 1 to 5 carbon atoms, or a substituted or unsubstituted phenyl group. n represents an integer of 1 to 5.

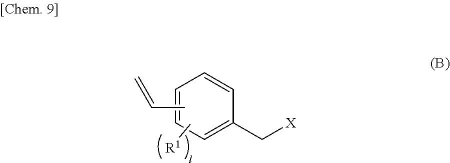

In the above formula (B), each of the multiple R₁ independently represents a hydrogen atom or a hydrocarbon group having 1 to 5 carbon atoms. Each of the multiple 1 independently represents an integer of 1 to 4. X represents a halogen atom.

### Advantageous Effects of Invention

According to the present invention, it is possible to provide a compound and a curable resin composition having excellent low dielectric properties.

### BRIEF DESCRIPTION OF DRAWINGS

FIG. 1 shows an HP-LC chart of Example 1.

FIG. 2 shows a ¹H-NMR chart of Example 1.

FIG. 3 shows an HP-LC chart of Example 2.

FIG. 4 shows a ¹H-NMR chart of Example 2.

FIG. 5 shows an HP-LC chart of Example 3.

FIG. 6 shows a ¹H-NMR chart of Example 3.

FIG. 7 shows an HP-LC chart of Example 4.

FIG. 8 shows a ¹H-NMR chart of Example 4.

FIG. 9 shows an HP-LC chart of Example 5.

FIG. 10 shows a ¹H-NMR chart of Example 5.

FIG. 11 shows an HP-LC chart of Example 6.

FIG. 12 shows a ¹H-NMR chart of Example 6.

FIG. 13 shows an HP-LC chart of Example 7.

FIG. 14 shows a ¹H-NMR chart of Example 7.

FIG. 15 shows an HP-LC chart of Example 8.

FIG. 16 shows a ¹H-NMR chart of Example 8.

FIG. 17 shows an HP-LC chart of Example 9.

FIG. 18 shows a ¹H-NMR chart of Example 9.

FIG. 19 shows an HP-LC chart of Example 10.

FIG. 20 shows a ¹H-NMR chart of Example 10.

FIG. 21 shows an HP-LC chart of Example 11.

FIG. 22 shows a ¹H-NMR chart of Example 11.

FIG. 23 shows an HP-LC chart of Example 12.

FIG. 24 shows a ¹H-NMR chart of Example 12.

FIG. 25 shows an HP-LC chart of Example 13.

FIG. 26 shows a ¹H-NMR chart of Example 13.

FIG. 27 shows an HP-LC chart of Example 14.

FIG. 28 shows a ¹H-NMR chart of Example 14.

FIG. 29 shows an HP-LC chart of Comparative Synthesis Example 1.

FIG. 30 shows a ¹H-NMR chart of Comparative Synthesis Example 1.

### DESCRIPTION OF EMBODIMENTS

Hereinafter, an embodiment of the present invention (hereinafter, also referred to as “the present embodiment”) will be described in further detail.

The compound of the present embodiment is represented by the following formula (1).

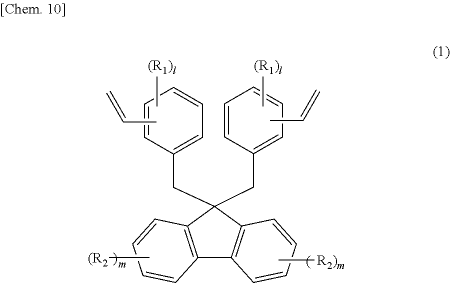

In the above formula (1), each of the multiple R₁ independently represents a hydrogen atom or a hydrocarbon group having 1 to 5 carbon atoms, preferably a hydrogen atom or a hydrocarbon group having 1 to 3 carbon atoms, and more preferably a hydrogen atom. When the carbon number is 5 or less, the molecular vibration is unlikely to occur when exposed to high frequency waves, and therefore the electrical properties are excellent. In addition, when the R₁ is a hydrogen atom, deterioration of the dielectric properties and water absorption properties due to the generation of polar groups derived from the oxidation reaction of the alkyl group during the high temperature storage test can be suppressed. Each of the multiple R 2s independently represents a hydrocarbon group represented by the following formula (a). Each of the multiple l's independently represents an integer of 1 to 4, preferably 1. Each of the multiple m's independently represents an integer of 0 to 4, and the average mₐᵥₑ of the values m is 0<mₐᵥₑ≤4. mₐᵥₑ may be calculated from the charging ratio of the raw materials, or may be calculated from the results of NMR analysis. From the viewpoint of solvent solubility and heat resistance, it is preferable that 0<mₐᵥₑ≤3, and more preferably 0<mₐᵥₑ≤2. The solvent solubility and dielectric properties are excellent when mₐᵥₑ is greater than 0, compared with the case where mₐᵥₑ=0. On the other hand, when mₐᵥₑ is greater than 4, steric hindrance becomes large, and there is a risk of reduced curability.

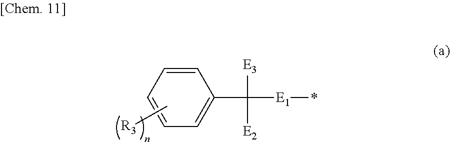

In the above formula (a), * represents the bonding position to the fluorene structure of formula (1). Each of the multiple R₃ independently represents a hydrogen atom or a hydrocarbon group having 1 to 5 carbon atoms, preferably a hydrogen atom or a hydrocarbon group having 1 to 3 carbon atoms, and more preferably a hydrogen atom. When the carbon number is 5 or less, the molecular vibration is unlikely to occur when exposed to high frequency waves, and therefore the electrical properties are excellent. In addition, when it is a hydrogen atom, it is possible to suppress the deterioration of the dielectric properties and water absorption properties associated with the generation of polar groups derived from the oxidation reaction of the alkyl group during the high temperature storage test. E₁ represents a direct bond or a methylene group. E₂ and E₃ represent a hydrogen atom, a hydrocarbon group having 1 to 5 carbon atoms, or a substituted or unsubstituted phenyl group, and the hydrocarbon group having 1 to 5 carbon atoms is preferably a methyl group. n represents an integer of 1 to 5, preferably 1.

The hydrocarbon group represented by the above formula (a) is preferably one or more types selected from the following formulae (a-1) to (a-6).

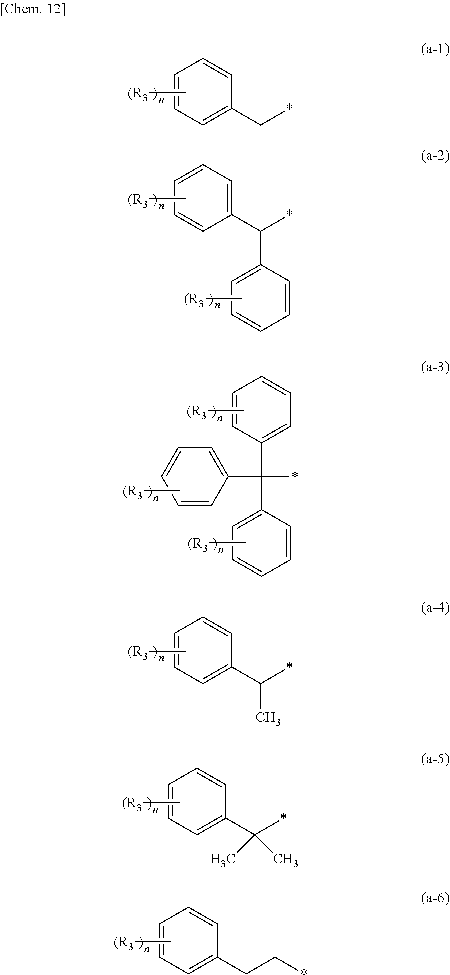

In the above formulas (a-1) to (a-6), * represents a bonding position to the fluorene structure of formula (1). Each of the multiple R₃ independently represents a hydrogen atom or a hydrocarbon group having 1 to 5 carbon atoms, preferably a hydrogen atom or a hydrocarbon group having 1 to 3 carbon atoms, and more preferably a hydrogen atom. When the number of carbon atoms is 5 or less, the molecular vibration is unlikely to occur when exposed to high frequency waves, and therefore the electrical properties are excellent. In addition, when the number is a hydrogen atom, deterioration of the dielectric properties and water absorption properties due to the generation of polar groups resulting from the oxidation reaction of the alkyl group during high temperature storage tests can be suppressed. Each of the multiple n's independently represents an integer of 1 to 5.

The above formulas (a-1) to (a-6) may be used alone or in combination. For example, the value (X/Y) obtained by dividing the number of introduced substituents (X) represented by the above formula (a-2) by the number of introduced substituents (Y) represented by the above formula (a-1) is preferably 0.25≤(X/Y)≤8, more preferably 0.5≤(X/Y)≤6, particularly preferably 0.75≤(X/Y)≤4, and most preferably 0.75≤(X/Y)≤2. The higher the content of the substituents represented by formula (a-2), the better the heat resistance, low water absorption, and low dielectric properties. The value of (X/Y) can be calculated from the charge ratio of the raw materials when synthesizing the compound represented by formula (A) described later.

The method for producing the compound represented by the above formula (1) is not particularly limited, but the compound can be obtained by reacting a compound represented by the following formula (A) with a compound represented by the following formula (B).

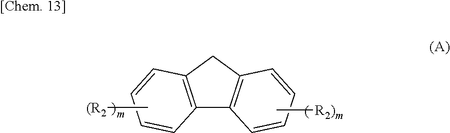

In the above formula (A), each of the multiple R₂ₛ independently represents a hydrocarbon group represented by the following formula (a). Each of the multiple m's independently represents an integer of 0 to 4. The average mₐᵥₑ of the values m is 0<mₐᵥₑ≤4. mₐᵥₑ may be calculated from the charging ratio of the raw materials, or may be calculated from the results of NMR analysis. From the viewpoint of solvent solubility and heat resistance, it is preferable that 0<mₐᵥₑ≤3.0, and more preferably 0<mₐᵥₑ≤2. The solvent solubility and dielectric properties are excellent when mₐᵥₑ is greater than 0 compared with the case where mₐᵥₑ=0. On the other hand, when mₐᵥₑ is greater than 4, steric hindrance becomes large, and there is a risk of curability decreasing.

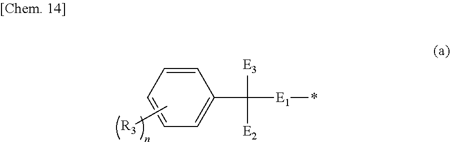

In the above formula (a), * represents the bonding position to the fluorene structure of formula (A). Each of the multiple R 3s independently represents a hydrogen atom or a hydrocarbon group having 1 to 5 carbon atoms, preferably a hydrogen atom or a hydrocarbon group having 1 to 3 carbon atoms, and more preferably a hydrogen atom. When the carbon number is 5 or less, the molecular vibration is unlikely to occur when exposed to high frequency waves, and therefore the electrical properties are excellent. In addition, when it is a hydrogen atom, it is possible to suppress the deterioration of the dielectric properties and water absorption properties associated with the generation of polar groups derived from the oxidation reaction of the alkyl group during the high temperature storage test. E₁ represents a direct bond or a methylene group. E₂ and E₃ represent a hydrogen atom, a hydrocarbon group having 1 to 5 carbon atoms, or a substituted or unsubstituted phenyl group, and the hydrocarbon group having 1 to 5 carbon atoms is preferably a methyl group. n represents an integer of 1 to 5, preferably 1.

The hydrocarbon group represented by the formula (a) is preferably one or more selected from the formulas (a-1) to (a-6), and the formulas (a-1) to (a-6) may be any one of them or a mixture of two or more. For example, the value (X/Y) obtained by dividing the number of introduced substituents (X) represented by the formula (a-2) by the number of introduced substituents (Y) represented by the formula (a-1) is preferably 0.25≤(X/Y)≤8, more preferably 0.5≤(X/Y)≤6, particularly preferably 0.75≤(X/Y)≤4, and most preferably 0.75≤(X/Y)≤2. The higher the content of the substituents represented by the formula (a-2), the better the heat resistance and low water absorption.

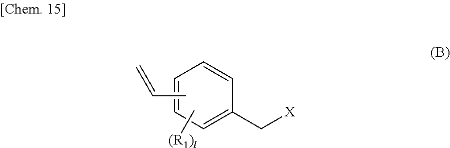

In the above formula (B), R₁ represents a hydrogen atom or a hydrocarbon group having 1 to 5 carbon atoms, preferably a hydrogen atom or a hydrocarbon group having 1 to 3 carbon atoms, and more preferably a hydrogen atom. When the carbon number is 5 or less, the molecular vibration is unlikely to occur when exposed to high frequency waves, and therefore the electrical properties are excellent. In addition, when it is a hydrogen atom, it is possible to suppress the deterioration of the dielectric properties and water absorption properties associated with the generation of polar groups resulting from the oxidation reaction of the alkyl group during the high temperature storage test. X represents a halogen atom, and from the viewpoint of reactivity and suppression of waste generation, it is preferably a bromine atom or a chlorine atom, and more preferably a chlorine atom. l represents an integer of 1 to 4, preferably 1.

In the case of synthesizing the compound represented by the formula (1), when the number of moles of the compound represented by the formula (A) is a and the number of moles of the compound represented by the formula (B) is B, B/a is preferably 1.8 to 2.1, more preferably 1.8 to 2.0 mol, and particularly preferably 1.8 to 1.95. When B/a is less than 1.8, the compound represented by the formula (A) remains unreacted, and the toughness of the cured film may decrease, and the dielectric properties may deteriorate. This is because the unreacted compound represented by the formula (A) does not have a structure that can be crosslinked, and the methylene structure at the 9th position of the compound represented by the formula (A) reacts with oxygen to generate a ketone, which increases the polarity. If/a is greater than 2.1, the halogen element of the compound represented by formula (B) that has not been completely removed by purification may be eliminated during curing (for example, at a temperature of 175° C. or higher) or during a high-temperature, high-humidity test (85° C., 85% humidity, 120° C., 100% humidity, etc.), which may lead to corrosion of the copper wiring. The amount of residual halogen contained in the compound of this embodiment is preferably 1 to 10,000 ppm, more preferably 1 to 3,000 ppm, and even more preferably 1 to 2,000 ppm.

The reaction between the compound represented by formula (A) and the compound represented by formula (B) will be described in detail. The compound represented by formula (1) is obtained by reacting the compound represented by formula (A) and the compound represented by formula (B) in an aprotic polar solvent in the presence of a basic catalyst. Examples of the aprotic polar solvent include dimethyl sulfone, dimethyl sulfoxide, dimethylformamide, dimethylacetamide, 1,3-dimethyl-2-imidazolidinone, and N-methylpyrrolidone, and two or more of them may be used in combination. In addition, a non-water-soluble solvent may be used in combination as necessary. Examples of the non-water-soluble solvent include, but are not limited to, aromatic solvents such as toluene and xylene, aliphatic solvents such as cyclohexane and n-hexane, ethers such as diethyl ether and diisopropyl ether, ester-based solvents such as ethyl acetate and butyl acetate, and ketone-based solvents such as methyl isobutyl ketone and cyclopentanone, and two or more of them may be used in combination. The catalyst is not particularly limited, and examples thereof include basic catalysts such as sodium hydroxide, potassium hydroxide, and potassium carbonate. The order of adding the compound represented by the formula (A), the compound represented by the formula (B), and the base can be changed as necessary, but a method of adding the compound represented by the formula (A), an aprotic polar solvent, and a base, sufficiently ionizing the compound represented by the formula (A), and then adding the compound represented by the formula (B) is preferred. When the reaction is carried out without using an aprotic polar solvent, the reaction rate is significantly reduced. When an aprotic polar solvent is not used, the reaction is generally carried out using an interlayer transfer catalyst. In this case, the raw material is dissolved in a non-aqueous solvent such as toluene, and the compound represented by the formula (A) and the compound represented by the formula (B) are reacted in the presence of a base catalyst such as an aqueous sodium hydroxide solution and an interlayer transfer catalyst such as tetrabutylammonium bromide. In this case, it is difficult to completely remove the interlayer transfer catalyst such as tetrabutylammonium bromide, making it difficult to achieve low dielectric properties (low dielectric constant and low dielectric tangent), and the remaining interlayer transfer catalyst may cause problems such as ion migration when the substrate material using the compound of this embodiment is subjected to a long-term wet heat reliability test or the like. The reaction temperature is preferably 0 to 120° C., more preferably 0 to 100° C., and even more preferably 0 to 80° C. If the temperature is above the upper limit, the compound of this embodiment may undergo self-polymerization and gelation. If the temperature is below the lower limit, the reaction may not proceed sufficiently. As a post-treatment after the reaction, neutralization may be performed with any acid compound. If necessary, an alcohol compound, water, or the like may be added to the reaction solution to recover the target product as crystals. The obtained reaction solution or crystals may be re-dissolved in any solvent to perform an extraction step. For the extraction step, an aromatic hydrocarbon solvent such as toluene or xylene may be used alone, or a non-aromatic hydrocarbon such as cyclohexane or toluene may be used in combination. After extraction, the organic layer is washed with water until the wastewater becomes neutral, and the target compound can be obtained by distilling off the solvent using an evaporator or the like.

The compound represented by the above formula (A) is derived from fluorene and a compound represented by the following formula (c) or (d).

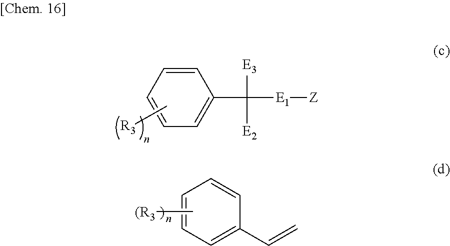

In the above formulas (c) and (d), the multiple R₃ each independently represent a hydrogen atom or a hydrocarbon group having 1 to 5 carbon atoms, preferably a hydrogen atom or a hydrocarbon group having 1 to 3 carbon atoms, and more preferably a hydrogen atom. When the carbon number is 5 or less, the molecular vibration is unlikely to occur when exposed to high frequency waves, and therefore the electrical properties are excellent. In addition, when the R₃ is a hydrogen atom, the deterioration of the dielectric properties and water absorption properties associated with the generation of polar groups derived from the oxidation reaction of the alkyl group during the high temperature storage test can be suppressed. The multiple n each independently represent an integer of 1 to 5, preferably 1. Z represents a hydroxy group or a halogen atom, and is preferably a hydroxy group, a bromine atom, or a chlorine atom from the viewpoint of reactivity and suppression of waste generation, and is more preferably a hydroxy group or a chlorine atom. E₁ represents a direct bond or a methylene group. E₂ and E₃ represent a hydrogen atom, a hydrocarbon group having 1 to 5 carbon atoms, or a substituted or unsubstituted phenyl group, and the hydrocarbon group having 1 to 5 carbon atoms is preferably a methyl group. Of the compounds represented by the above formula (c) or (d), it is preferable to use the compound represented by the above formula (c) from the viewpoint of suppressing the self-polymerization reaction of the raw material.

The compound represented by the above formula (c) is preferably selected from the following formulas (c-1) to (c-6):

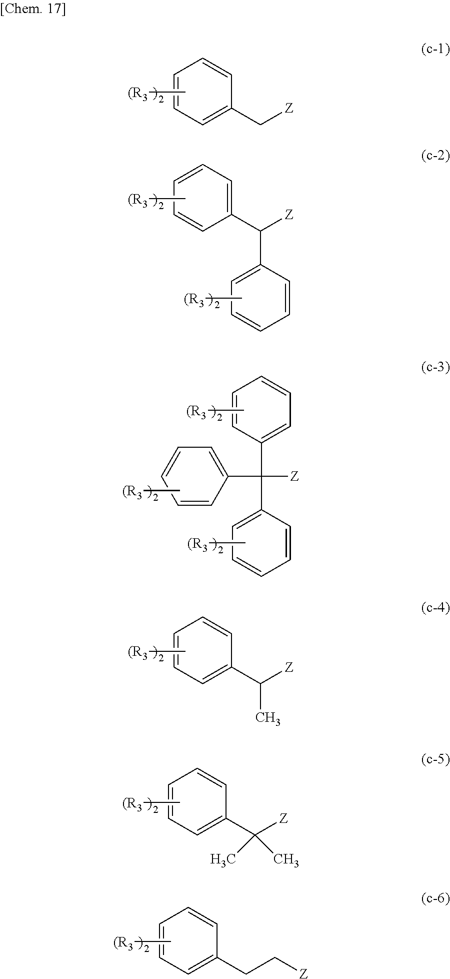

When reacting fluorene with a compound represented by the above formula (c) or (d), it is preferable to react 0.01 to 10 mol of the compound represented by the above formula (c) or (d) with 1 mol of fluorene, more preferably 0.1 to 7.5 mol, even more preferably 0.2 to 5 mol, and most preferably 0.3 to 4 mol. If the number of moles of the compound (c) or (d) reacted with fluorene is less than 0.01 mol, there is a concern that the storage stability after dissolving in a solvent may deteriorate, such as crystals being precipitated when left for a long time after dissolving in a solvent, and if it is greater than 10 mol, there is a risk that the heat resistance may decrease, or the compound represented by the above formula (c) or (d) may become excessive, self-polymerize, and the purity of the target product may decrease.

When reacting fluorene with the compound represented by the above formula (c) or (d), as necessary, hydrochloric acid, phosphoric acid, sulfuric acid, formic acid, p-toluenesulfonic acid, methanesulfonic acid; Lewis acids such as aluminum chloride and zinc chloride; activated clay, acidic clay, white carbon, zeolite, solid acids such as silica alumina, acidic ion exchange resins, etc. can be used as a catalyst. These may be used alone or in combination of two or more. The amount of catalyst used is preferably 0.1 to 40% by weight, more preferably 0.1 to 20% by weight, based on the total weight of fluorene and the compound represented by the above formula (c) or (d). If the amount of catalyst used is too large, the viscosity of the reaction solution may be too high, making stirring difficult, and if the amount is too small, the reaction may proceed slowly. The reaction may be carried out using an organic solvent such as hexane, cyclohexane, octane, toluene, or xylene, as necessary, or may be carried out without a solvent. For example, after adding an acid catalyst to a mixed solution of fluorene, a compound represented by the above formula (c) or (d), and a solvent (or no solvent), if the catalyst contains water, the water is removed from the system by azeotropy or the like. Then, the reaction is carried out at 40 to 300° C., preferably 50 to 250° C., for 0.5 to 40 hours. After the reaction is completed, the acid catalyst may be neutralized with an alkaline aqueous solution, but it is also possible to proceed to the water washing step without neutralization. In the water washing step, a water-insoluble organic solvent is added to the oil layer and water washing is repeated until the wastewater becomes neutral.

In addition, the compound represented by formula (A) obtained by the above reaction may be continuously derived into the compound represented by formula (1) by adding a neutralizing agent such as an alkali and an aprotic polar solvent to the solution after the reaction, and then adding a base catalyst and the compound represented by formula (B).

The softening point of the compound represented by formula (A) is preferably 150° C. or less, more preferably 120° C. or less. When the softening point is 150° C. or less, the viscosity when induced into the compound represented by formula (1) is low. This makes it easy to ensure fluidity, does not impregnate glass cloth or carbon fiber, and facilitates B-stage formation such as prepreg formation. If the viscosity is reduced by increasing the dilution solvent, there is a possibility that the resin will not sufficiently adhere to the fibrous material in the impregnation process.

The compound represented by the above formula (1) can be cured by itself by heating or the like, but performance can also be improved by adding various materials to form a curable resin composition.

### [Curing Accelerator]

The curing property of the curable resin composition of the present embodiment can be improved by adding a curing accelerator. As the curing accelerator, an anionic curing accelerator that accelerates the curing reaction by generating anions upon irradiation with ultraviolet light or visible light or heating, or a cationic curing accelerator that accelerates the curing reaction by generating cations upon irradiation with ultraviolet light or visible light or heating, is preferable.

Examples of the anionic curing accelerator include imidazoles such as 2-methylimidazole, 2-ethylimidazole, and 2-ethyl-4-methylimidazole, trialkylamines such as triethylamine and tributylamine, 4-dimethylaminopyridine, benzyldimethylamine, 2,4,6-tris(dimethylaminomethyl) phenol, and 1,8-diazabicyclo(5,4,0)-undecene, with 4-dimethylaminopyridine and 1,8-diazabicyclo(5,4,0)-undecene being preferred. Other examples include phosphines such as triphenylphosphine; quaternary ammonium salts such as tetrabutylammonium salts, triisopropylmethylammonium salts, trimethyldecanylammonium salts, cetyltrimethylammonium salts, and hexadecyltrimethylammonium hydroxide, but are not limited thereto. These may be used alone or in combination.

Examples of the cationic curing accelerator include quaternary phosphonium salts such as triphenylbenzylphosphonium salt, triphenylethylphosphonium salt, and tetrabutylphosphonium salt (the counter ion of the quaternary salt may be a halogen, an organic acid ion, a hydroxide ion, or the like, and is not particularly specified, but an organic acid ion or a hydroxide ion is particularly preferred); transition metal compounds (transition metal salts) such as tin octylate, zinc carboxylate (zinc 2-ethylhexanoate, zinc stearate, zinc behenate, zinc myristate), and zinc phosphate (zinc octylphosphate, zinc stearylphosphate), but are not limited thereto. Furthermore, these may be used alone or in combination.

The curing accelerator is used in an amount of 0.01 to 5.0 parts by mass based on 100 parts by mass of the curable resin composition, as required.

### [Inorganic Filler]

The curable resin composition of the present embodiment may contain an inorganic filler. Examples of inorganic fillers include powders such as fused silica, crystalline silica, porous silica, alumina, zircon, calcium silicate, calcium carbonate, quartz powder, silicon carbide, silicon nitride, boron nitride, zirconia, aluminum nitride, graphite, forsterite, steatite, spinel, mullite, titania, talc, clay, iron oxide, asbestos, and glass powder, and inorganic fillers obtained by making these into a spherical or crushed shape, but are not limited thereto. In addition, these may be used alone or in combination.

When a curable resin composition for semiconductor encapsulation is obtained, the amount of the inorganic filler used is preferably 80 to 92 parts by mass, and more preferably 83 to 90 parts by mass, based on 100 parts by mass of the curable resin composition. When a curable resin composition for an interlayer insulating layer forming material, or a substrate material such as a copper-clad laminate, prepreg, or RCC is obtained, the amount of the inorganic filler used is preferably 5 to 80 parts by mass, and more preferably 10 to 60 parts by mass, based on 100 parts by mass of the curable resin composition.

### [Polymerization Initiator]

The curable resin composition of the present embodiment can also improve the curability by adding a polymerization initiator. The polymerization initiator is a compound capable of polymerizing an olefin functional group such as an ethylenically unsaturated bond, and examples of the polymerization initiator include an olefin metathesis polymerization initiator, an anionic polymerization initiator, a cationic polymerization initiator, and a radical polymerization initiator. Among these, it is preferable to use a radical polymerization initiator having curability and moderate stability. The radical polymerization initiator is a compound that generates radicals by irradiation with ultraviolet light or visible light or by heating, and starts a chain polymerization reaction. Examples of radical polymerization initiators that can be used include organic peroxides, azo compounds, and benzopinacoles, and it is preferable to use an organic peroxide because it has little effect on curing temperature control, outgassing suppression, and electrical properties of decomposition products.

Examples of the organic peroxides include, but not limited to: ketone peroxides such as methyl ethyl ketone peroxide and acetylacetone peroxide; diacyl peroxides such as benzoyl peroxide; dialkyl peroxides such as dicumyl peroxide and 1,3-bis-(t-butylperoxyisopropyl)-benzene; peroxyketals such as t-butylperoxybenzoate and 1,1-di-t-butylperoxycyclohexane; alkyl peresters such as α-cumylperoxyneodecanoate, t-butylperoxyneodecanoate, t-butylperoxypivalate, 1,1,3,3-tetramethylbutylperoxy-2-ethylhexanoate, t-amylperoxy-2-ethylhexanoate; peroxycarbonates such as di-2-ethylhexyl peroxydicarbonate, bis(4-t-butylcyclohexyl) peroxydicarbonate, t-butylperoxyisopropyl carbonate, 1,6-bis(t-butylperoxycarbonyloxy) hexane; t-butyl hydroperoxide, cumene hydroperoxide, t-butyl peroxyoctoate, lauroyl peroxide, etc. These may be used alone or in combination. Among the above organic peroxides, ketone peroxides, diacyl peroxides, hydroperoxides, dialkyl peroxides, peroxyketals, alkyl peresters, peroxycarbonates, etc. are preferred, with dialkyl peroxides being more preferred.

Examples of the azo-based compound include, but are not limited to, azobisisobutyronitrile, 4,4′-azobis(4-cyanovaleric acid), 2,2′-azobis(2,4-dimethylvaleronitrile), etc. These may be used alone or in combination.

The amount of the polymerization initiator added is preferably 0.01 to 5 parts by mass, and particularly preferably 0.01 to 3 parts by mass, relative to 100 parts by mass of the curable resin composition. If the amount of the polymerization initiator used is less than 0.01 part by mass, the molecular weight may not be sufficiently extended during the polymerization reaction, and if it is more than 5 parts by mass, the dielectric properties such as the dielectric constant and the dielectric loss tangent may be impaired.

### [Polymerization Inhibitor]

The curable resin composition of the present embodiment may contain a polymerization inhibitor. By containing a polymerization inhibitor, storage stability is improved and the reaction initiation temperature can be controlled. By controlling the reaction initiation temperature, it becomes easy to ensure fluidity, impregnation into glass cloth and the like is not impaired, and B-stage such as prepreg formation is facilitated. If the polymerization reaction proceeds too much during prepreg formation, problems such as difficulty in lamination during the lamination process are likely to occur.

The polymerization inhibitor may be added during or after the synthesis of the compound of this embodiment. The amount of the polymerization inhibitor used is 0.008 to 1 part by mass, preferably 0.01 to 0.5 parts by mass, based on 100 parts by weight of the compound of this embodiment.

Examples of the polymerization inhibitor include phenol-based, sulfur-based, phosphorus-based, hindered amine-based, nitroso-based, and nitroxyl radical-based. The polymerization inhibitor may be used alone or in combination. Among these, in the present embodiment, the phenol-based, hindered amine-based, nitroso-based, and nitroxyl radical-based are preferred.

Examples of the phenol-based polymerization inhibitor include, but are not limited to: monophenols such as 2,6-di-t-butyl-p-cresol, butylated hydroxyanisole, 2,6-di-t-butyl-p-ethylphenol, stearyl-β-(3,5-di-t-butyl-4-hydroxyphenyl) propionate, isooctyl-3-(3,5-di-t-butyl-4-hydroxyphenyl) propionate, 2,4-bis-(n-octylthio)-6-(4-hydroxy-3,5-di-t-butylanilino)-1,3,5-triazine, and 2,4-bis[(octylthio)methyl]-o-cresol; bisphenols such as 2,2′-methylenebis(4-methyl-6-t-butylphenol), 2,2′-methylenebis(4-ethyl-6-t-butylphenol), 4,4′-thiobis(3-methyl-6-t-butylphenol), 4,4′-butylidenebis(3-methyl-6-t-butylphenol), triethylene glycol-bis[3-(3-t-butyl-5-methyl-4-hydroxyphenyl) propionate], 1,6-hexanediol-bis[3-(3,5-di-t-butyl-4-hydroxyphenyl) propionate], N,N′-hexamethylenebis(3,5-di-t-butyl-4-hydroxy-hydrocinnamamide), 2,2-thio-diethylenebis[3-(3,5-di-t-butyl-4-hydroxyphenyl)proponate], 3,5-di-t-butyl-4-hydroxybenzylphosphanate-diethylester, 3,9-bis[1,1-dimethyl 2-{β-(3-t-butyl-4-hydroxy-5-methylphenyl) propionyloxy}ethyl]2,4,8,10-tetraoxaspiro[5,5]undecane,bis(3,5-di-t-butyl-4-hydroxybenzylsulfonate ethyl) calcium; polymeric phenols such as 1,1,3-tris(2-methyl-4-hydroxy-5-t-butylphenyl) butane, 1,3,5-trimethyl-2,4,6-tris(3,5-di-t-butyl-4-hydroxybenzyl)benzene, tetrakis-[methylene-3-(3′,5′-di-t-butyl-4′-hydroxyphenyl) propionate]methane, bis[3,3′-bis-(4′-hydroxy-3′-t-butylphenyl) butyric acid]glycol ester, tris-(3,5-di-t-butyl-4-hydroxybenzyl)-isocyanurate, 1,3,5-tris(3′,5′-di-t-butyl-4′-hydroxybenzyl)-s-triazine-2,4,6-(1H,3H,5H)trione, and tocopherol.

Examples of the sulfur-based polymerization inhibitor include, but are not limited to, dilauryl-3,3′-thiodipropionate, dimyristyl-3,3′-thiodipropionate, and distearyl-3,3′-thiodipropionate.

Examples of the phosphorus-based polymerization inhibitor include, but are not limited to: phosphites such as triphenyl phosphite, diphenyl isodecyl phosphite, phenyl diisodecyl phosphite, tris(nonylphenyl) phosphite, diisodecyl pentaerythritol phosphite, tris(2,4-di-t-butylphenyl) phosphite, cyclic neopentane tetrayl bis(octadecyl)phosphite, cyclic neopentane tetrayl bis(2,4-di-t-butylphenyl) phosphite, cyclic neopentane tetrayl bis(2,4-di-t-butyl-4-methylphenyl) phosphite, bis[2-t-butyl-6-methyl-4-{2-(octadecyloxycarbonyl)ethyl}phenyl]hydrogen phosphite; and oxaphosphaphenanthrene oxides such as 9,10-dihydro-9-oxa-10-phosphaphenanthrene-10-oxide, 10-(3,5-di-t-butyl-4-hydroxybenzyl)-9,10-dihydro-9-oxa-10-phosphaphenanthrene-10-oxide, and 10-decyloxy-9,10-dihydro-9-oxa-10-phosphaphenanthrene-10-oxide.

Examples of the hindered amine-based polymerization inhibitor include, but are not limited to: ADK STAB (registered trademark) LA-40MP, ADK STAB LA-40Si, ADK STAB LA-402AF, ADK STAB LA-87, ADK STAB LA-82, ADK STAB LA-81, ADK STAB LA-77Y, ADK STAB LA-77G, ADK STAB LA-72, ADK STAB LA-68, ADK STAB LA-63P, ADK STAB LA-57, and ADK STAB LA-52 (all manufactured by ADEKA Corporation); Chimassorb (registered trademark) 2020FDL, Chimassorb 944FDL, Chimassorb 944LD, Tinuvin (registered trademark) 622SF, Tinuvin PA144, Tinuvin 765, Tinuvin 770DF, Tinuvin XT55FB, Tinuvin 111FDL, Tinuvin 783FDL, and Tinuvin 791FB (all manufactured by BASF Corporation).

Examples of the nitroso-based polymerization inhibitor include, but are not limited to, p-nitrosophenol, N-nitrosodiphenylamine, ammonium salt of N-nitrosophenylhydroxyamine (cupferron), etc. Among these, the ammonium salt of N-nitrosophenylhydroxyamine (cupferron) is preferred.

Examples of the nitroxyl radical polymerization inhibitor include, but are not limited to: di-tert-butyl nitroxide, 2,2,6,6-tetramethylpiperidine-1-oxyl, 4-hydroxy-2,2,6,6-tetramethylpiperidine-1-oxyl, 4-oxo-2,2,6,6-tetramethylpiperidine-1-oxyl, 4-amino-2,2,6,6-tetramethylpiperidine-1-oxyl, 4-methoxy-2,2,6,6-tetramethylpiperidine-1-oxyl, 4-acetoxy-2,2,6,6-tetramethylpiperidine-1-oxyl, 4-benzoyloxy-2,2,6,6-tetramethylpiperidine-1-oxyl, and the like.

### [Flame Retardant]

The curable resin composition of the present embodiment may contain a flame retardant. Examples of the flame retardant include halogen-based flame retardants, inorganic flame retardants (antimony compounds, metal hydroxides, nitrogen compounds, boron compounds, etc.), and phosphorus-based flame retardants. From the viewpoint of achieving halogen-free flame retardancy, phosphorus-based flame retardants are preferred.

The phosphorus-based flame retardant may be of a reactive type or an additive type. Specific examples include phosphoric acid esters such as trimethyl phosphate, triethyl phosphate, tricresyl phosphate, trixylyleneyl phosphate, cresyl diphenyl phosphate, cresyl-2,6-dixylyleneyl phosphate, 1,3-phenylene bis(dixylyleneyl phosphate), 1,4-phenylene bis(dixylyleneyl phosphate), and 4,4′-biphenyl(dixylyleneyl phosphate), phosphanes such as 9,10-dihydro-9-oxa-10-phosphaphenanthrene-10-oxide and 10-(2,5-dihydroxyphenyl)-10H-9-oxa-10-phosphaphenanthrene-10-oxide, phosphorus-containing epoxy compounds obtained by reacting epoxy resins with active hydrogen of the above-mentioned phosphanes, red phosphorus, and the like, but are not limited thereto. In addition, these may be used alone or in combination. Of the above-listed substances, phosphates, phosphanes, or phosphorus-containing epoxy compounds are preferred, with 1,3-phenylenebis(dixylilenyl phosphate), 1,4-phenylenebis(dixylilenyl phosphate), 4,4′-biphenyl(dixylilenyl phosphate) or phosphorus-containing epoxy compounds being particularly preferred.

The content of the flame retardant is preferably in the range of 0.1 to 0.6 parts by mass relative to 100 parts by mass of the curable resin composition. If the content is less than 0.1 part by mass, the flame retardancy may be insufficient, and if the content is more than 0.6 part by mass, the moisture absorption and dielectric properties of the cured product may be adversely affected.

### [Light Stabilizer]

The curable resin composition of the present embodiment may contain a light stabilizer. As the light stabilizer, a hindered amine light stabilizer (Hindered Amine Light Stabilizers, HALS) or the like is preferable. Examples of HALS include, but are not limited to: a reaction product of dibutylamine, 1,3,5-triazine, N,N′-bis(2,2,6,6-tetramethyl-4-piperidyl)-1,6-hexamethylenediamine and N-(2,2,6,6-tetramethyl-4-piperidyl)butylamine; a reaction product of dimethyl succinate-1-(2-hydroxyethyl)-4-hydroxy-2,2,6,6-tetramethylpiperidine; and poly[{6-(1,1,3,3-tetramethylbutyl)amino-1,3,5-triazine-2,4-diyl}{(2,2,6,6-tetramethyl-4-piperidyl)imino}hexamethylene {(2,2,6,6-tetramethyl-4-piperidyl)imino}], bis(1,2,2,6,6-pentamethyl-4-piperidyl) [[3,5-bis(1,1-dimethylethyl)-4-hydroxyphenyl]methyl]butyl malonate, bis(2,2,6,6-tetramethyl-4-piperidyl) sebacate, bis(1,2,2,6,6-pentamethyl-4-piperidyl) sebacate, bis(1-octyloxy-2,2,6,6-tetramethyl-4-piperidyl) sebacate, 2-(3,5-di-t-butyl-4-hydroxybenzyl)-2-n-butylmalonate bis(1,2,2,6,6-pentamethyl-4-piperidyl), and the like. These may be used alone or in combination.

The content of the light stabilizer is preferably in the range of 0.001 to 0.1 parts by mass relative to 100 parts by mass of the curable resin composition. If the content is less than 0.001 part by mass, the light stabilizing effect may be insufficient, and if the content is more than 0.1 part by mass, the moisture absorption and dielectric properties of the cured product may be adversely affected.

### [Binder Resin]

The curable resin composition of the present embodiment may use a binder resin. Examples of binder resins include, but are not limited to, butyral resins, acetal resins, acrylic resins, epoxy-nylon resins, NBR-Phenolic resins, epoxy-NBR resins, and silicone resins. These may be used alone or in combination.

The amount of the binder resin to be blended is preferably within a range that does not impair the flame retardancy and heat resistance of the cured product, and is preferably 0.05 to 50 parts by mass, and more preferably 0.05 to 20 parts by mass, based on 100 parts by mass of the curable resin composition, as needed.

### [Additives]

The curable resin composition of the present embodiment may contain additives. Examples of the additives includes: modified acrylonitrile copolymers, polyethylene, fluororesins, silicone gels, silicone oils, surface treatment agents for fillers such as silane coupling agents, release agents, and colorants such as carbon black, phthalocyanine blue, and phthalocyanine green.

The amount of the additive is preferably 30 parts by mass or less, more preferably 20 parts by mass or less, and particularly preferably 10 parts by mass or less, based on 100 parts by mass of the curable resin composition.

The curable resin composition of the present embodiment may further include epoxy resins, active ester compounds, phenolic resins, polyphenylene ether compounds, amine resins, compounds having ethylenic unsaturated bonds, isocyanate resins, polyamide resins, maleimide compounds, cyanate ester resins, polyimide resins, polybutadiene and modified products thereof, polystyrene and modified products thereof, polyethylene and modified products thereof, etc., which may be used alone or in combination. Of these compounds, it is preferable to contain polyphenylene ether compounds, compounds having ethylenic unsaturated bonds, cyanate ester resins, polybutadiene and modified products thereof, and polystyrene and modified products thereof, in view of the balance of heat resistance, adhesion, and dielectric properties. By containing these compounds, the brittleness of the cured product can be improved and adhesion to metals can be improved, and cracks in the package during reliability tests such as solder reflow and thermal cycles can be suppressed.

Unless otherwise specified, the amount of the above compounds used is preferably times by mass or less, more preferably 5 times by mass or less, and particularly preferably 3 times by mass or less, relative to the compound of this embodiment. The preferred lower limit is 0.1 times by mass or more, more preferably 0.25 times by mass or more, and even more preferably 0.5 times by mass or more. By being within the above range, the effect of each compound added can be added while taking advantage of the effect of the low dielectric property of the compound of this embodiment. As these components, the following examples can be used.

### [Epoxy Resin]

Preferred examples of the epoxy resin are shown below, but are not limited to these. The epoxy resin may be liquid or solid, and may be used alone or in combination.

Examples of liquid epoxy resins include: bisphenol A type epoxy resins, bisphenol F type epoxy resins, bisphenol AF type epoxy resins, naphthalene type epoxy resins, glycidyl ester type epoxy resins, glycidyl amine type epoxy resins, phenol novolac type epoxy resins, alicyclic epoxy resins having an ester skeleton, cyclohexane type epoxy resins, cyclohexane dimethanol type epoxy resins, glycidyl amine type epoxy resins, and epoxy resins having a butadiene structure. Specific examples include “RE310S”, “RE410S” (all manufactured by Nippon Kayaku Co., Ltd., bisphenol A type epoxy resin), “RE303S”, “RE304S”, “RE403S”, “RE404S” (all manufactured by Nippon Kayaku Co., Ltd., bisphenol F type epoxy resin), “HP4032”, “HP4032D”, “HP4032SS” (all manufactured by DIC Corporation, naphthalene type epoxy resin), “828US”, “jER828EL”, “825”, “828EL” (all manufactured by Mitsubishi Chemical Corporation, bisphenol A type epoxy resin), “jE807”, “1750” (all manufactured by Mitsubishi Chemical Corporation, bisphenol F type epoxy resin), “jER (registered trademark) 152” (manufactured by Mitsubishi Chemical Corporation, phenol novolac type epoxy resin), “630”, “630LSD” (all manufactured by Mitsubishi Chemical Corporation, glycidylamine type epoxy resin), “ZX1059” (manufactured by Nippon Steel & Sumitomo Metal Chemical Co., Ltd., a mixture of bisphenol A type epoxy resin and bisphenol F type epoxy resin), “EX-721” (manufactured by Nagase ChemteX Corporation, glycidyl ester type epoxy resin), “Celloxide 2021P” (manufactured by Daicel Corporation, alicyclic epoxy resin having an ester skeleton), “PB-3600” (manufactured by Daicel Corporation, epoxy resin having a butadiene structure), “ZX1658”, “ZX1658GS” (all manufactured by Nippon Steel & Sumitomo Metal Chemical Co., Ltd., liquid 1,4-glycidylcyclohexane type epoxy resin), and the like. These may be used alone or in combination of two or more.

Preferred examples of solid epoxy resins include: bixylenol type epoxy resins, naphthalene type epoxy resins, naphthalene type tetrafunctional epoxy resins, cresol novolac type epoxy resins, dicyclopentadiene type epoxy resins, trisphenol type epoxy resins, naphthol type epoxy resins, biphenyl type epoxy resins, naphthylene ether type epoxy resins, anthracene type epoxy resins, bisphenol A type epoxy resins, bisphenol AF type epoxy resins, and tetraphenylethane type epoxy resins, and examples of such solid epoxy resins include naphthol type epoxy resins, bisphenol AF type epoxy resins, naphthalene type epoxy resins, and biphenyl type epoxy resins. Specific examples include “HP4032H” (manufactured by DIC Corporation, naphthalene type epoxy resin), “HP-4700”, and “HP-4710” (all manufactured by DIC Corporation, naphthalene type tetrafunctional epoxy resin), “N-690” (manufactured by DIC Corporation, cresol novolac type epoxy resin), “N-695” (manufactured by DIC Corporation, cresol novolac type epoxy resin), “HP-7200”, “HP-7200HH”, and “HP-7200H” (all manufactured by DIC Corporation, dicyclopentadiene type epoxy resin), “EXA-7311”, “EXA-7311-G3”, “EXA-7311-G4”, “EXA-7311-G4S”, “HP-6000” (all manufactured by DIC Corporation, naphthylene ether type epoxy resin), “EPPN-502H” (manufactured by Nippon Kayaku Co., Ltd., trisphenol type epoxy resin), “NC-7000L”, “NC-7300” (all manufactured by Nippon Kayaku Co., Ltd., naphthol-cresol novolac type epoxy resin), “NC-3000H”, “NC-3000”, “NC-3000L”, “NC-3100” (all manufactured by Nippon Kayaku Co., Ltd., biphenyl ether type epoxy resin), “XD-1000-2L”, “XD-1000-L”, “XD-1000-H” (all manufactured by Nippon Kayaku Co., Ltd., dicyclopentadiene type epoxy resin), “ESN475V” (manufactured by Nippon Steel & Sumikin Chemical Co., Ltd., naphthol type epoxy resin), “ESN485” (manufactured by Nippon Steel & Sumikin Chemical Co., Ltd., naphthol novolac type epoxy resin), “YX-4000H”, “YX-4000”, “YL6121” (all manufactured by Mitsubishi Chemical Corporation, biphenyl type epoxy resin), “YX-4000HK” (manufactured by Mitsubishi Chemical Corporation, bixylenol type epoxy resin), “YX-8800” (manufactured by Mitsubishi Chemical Corporation, anthracene type epoxy resin), “PG-100”, “CG-500” (manufactured by Osaka Gas Chemical Co., Ltd., fluorene type epoxy resin), “YL-7760” (manufactured by Mitsubishi Chemical Corporation, bisphenol AF type epoxy resin), “YL-7800” (manufactured by Mitsubishi Chemical Corporation, fluorene type epoxy resin), “jER1010” (manufactured by Mitsubishi Chemical Corporation, solid bisphenol A type epoxy resin), “jER1031S” (manufactured by Mitsubishi Chemical Corporation, tetraphenylethane type epoxy resin), etc. These may be used alone or in combination of two or more.

### [Active Ester Compound]

The active ester compound refers to a compound that contains at least one ester bond in the structure and has an aliphatic chain, an aliphatic ring, or an aromatic ring bonded to both sides of the ester bond. Examples of the active ester compound include compounds having two or more highly reactive ester groups in one molecule, such as phenol esters, thiophenol esters, N-hydroxyamine esters, and esters of heterocyclic hydroxy compounds, and are obtained by a condensation reaction between at least one compound of a carboxylic acid compound, an acid chloride, or a thiocarboxylic acid compound and at least one compound of a hydroxy compound or a thiol compound. In particular, from the viewpoint of improving heat resistance, it is preferable to obtain the active ester compound from a carboxylic acid compound or an acid chloride and a hydroxy compound, and the hydroxy compound is preferably a phenol compound or a naphthol compound. The active ester compound may be used alone or in combination of two or more types.

Examples of the carboxylic acid compound include benzoic acid, acetic acid, succinic acid, maleic acid, itaconic acid, phthalic acid, isophthalic acid, terephthalic acid, and pyromellitic acid.

Examples of the acid chloride include acetyl chloride, acrylic acid chloride, methacrylic acid chloride, malonyl chloride, succinic acid dichloride, diglycolyl chloride, glutaric acid dichloride, suberic acid dichloride, sebacic acid dichloride, adipic acid dichloride, dodecandioyl dichloride, azelaic acid chloride, 2,5-furandicarbonyl dichloride, phthaloyl chloride, isophthaloyl chloride, terephthaloyl chloride, trimesic acid chloride, bis(4-chlorocarbonylphenyl) ether, 4,4′-diphenyldicarbonyl chloride, and 4,4′-azodibenzoyl dichloride.

Examples of the phenol compound and naphthol compound include hydroquinone, resorcin, bisphenol A, bisphenol F, bisphenol S, phenolphthaline, methylated bisphenol A, methylated bisphenol F, methylated bisphenol S, phenol, o-cresol, m-cresol, p-cresol, catechol, α-naphthol, β-naphthol, 1,5-dihydroxynaphthalene, 1,6-dihydroxynaphthalene, 2,6-dihydroxynaphthalene, dihydroxybenzophenone, trihydroxybenzophenone, tetrahydroxy benzophenone, phloroglucin, benzenetriol, dicyclopentadiene-type diphenol compound, phenol novolak, and Phenolic resins described below. Here, the term “dicyclopentadiene-type diphenol compound” refers to a diphenol compound obtained by condensing one molecule of dicyclopentadiene with two molecules of phenol.

Preferred specific examples of the active ester compound include an active ester compound containing a dicyclopentadiene-type diphenol structure, an active ester compound containing a naphthalene structure, an active ester compound containing an acetylated product of phenol novolac, an active ester compound containing a benzoylated product of phenol novolac, a compound described in Example 2 of WO 2020/095829, and a compound disclosed in WO 2020/059625. Among them, an active ester compound containing a naphthalene structure and an active ester compound containing a dicyclopentadiene-type diphenol structure are more preferred. The dicyclopentadiene-type diphenol structure refers to a divalent structural unit consisting of phenylene-dicyclopentylene-phenylene.

Commercially available active ester compounds include, for example: active ester compounds containing a dicyclopentadiene-type diphenol structure such as “EXB9451”, “EXB9460”, “EXB9460S”, “HPC-8000-65T”, “HPC-8000H-65TM”, “EXB-8000L-65TM”, and “EXB-8150-65T” (manufactured by DIC Corporation); active ester compounds containing a naphthalene structure such as “EXB9416-70BK” (manufactured by DIC Corporation); active ester compounds containing an acetylated phenol novolac such as “DC808” (manufactured by Mitsubishi Chemical Corporation); active ester compounds containing a benzoylated phenol novolac such as “YLH1026”, “YLH1030”, and “YLH1048” (manufactured by Mitsubishi Chemical Corporation); an active ester curing agent which is an acetylated phenol novolac such as “DC808” (manufactured by Mitsubishi Chemical Corporation); and an active ester curing agent containing a phosphorus atom such as “EXB-9050L-62M” (manufactured by DIC Corporation).

Regarding the compounding ratio of the active ester compound and the epoxy resin, the ratio (α/β) of the active ester equivalent (α) to the epoxy equivalent (β) is preferably 0.5 to 1.5, more preferably 0.8 to 1.2, and even more preferably 0.90 to 1.10. If it is out of the above range, there is a risk that excess epoxy groups or active ester groups will remain in the system, and the characteristics may deteriorate in a high-temperature storage test (e.g., 150° C., 1000 hours) or a long-term reliability test under high-temperature and high-humidity conditions (e.g., temperature: 85° C., humidity: 85%).

### [Phenolic Resin]

Phenolic resin is a compound having two or more phenolic hydroxy groups in the molecule. Examples of Phenolic resin include, but are not limited to: reaction products of phenols and aldehydes, reaction products of phenols and diene compounds, reaction products of phenols and ketones, reaction products of phenols and substituted biphenyls, reaction products of phenols and substituted phenyls, reaction products of bisphenols and aldehydes, etc. Furthermore, these may be used alone or in combination.

Specific examples of the above raw materials are shown below, but are not limited to these.
<Phenols> Phenol, alkyl-substituted phenol, aromatic-substituted phenol, hydroquinone, resorcin, naphthol, alkyl-substituted naphthol, dihydroxybenzene, alkyl-substituted dihydroxybenzene, dihydroxynaphthalene, etc.
<Aldehydes> Formaldehyde, acetaldehyde, alkyl aldehyde, benzaldehyde, alkyl-substituted benzaldehyde, hydroxybenzaldehyde, naphthaldehyde, glutaraldehyde, phthalaldehyde, crotonaldehyde, cinnamaldehyde, furfural, etc.
<Diene compounds> Dicyclopentadiene, terpenes, vinylcyclohexene, norbornadiene, vinylnorbornene, tetrahydroindene, divinylbenzene, divinylbiphenyl, diisopropenylbiphenyl, butadiene, isoprene, etc.
<Ketones> Acetone, methyl ethyl ketone, methyl isobutyl ketone, acetophenone, benzophenone, fluorenone, etc.
<Substituted biphenyls> 4,4′-bis(chloromethyl)-1,1′-biphenyl, 4,4′-bis(methoxymethyl)-1,1′-biphenyl, 4,4′-bis(hydroxymethyl)-1,1′-biphenyl, etc.
<Substituted phenyls> 1,4-bis(chloromethyl)benzene, 1,4-bis(methoxymethyl)benzene, 1,4-bis(hydroxymethyl)benzene, etc.

### [Polyphenylene Ether Compound]

From the viewpoint of heat resistance and electrical properties, the polyphenylene ether compound is preferably a polyphenylene ether compound having an ethylenically unsaturated bond, and more preferably a polyphenylene ether compound having an acrylic group, a methacrylic group, or a styrene structure. Commercially available products include SA-9000 (manufactured by SABIC, a polyphenylene ether compound having a methacrylic group) and OPE-2St-1200 (manufactured by Mitsubishi Gas Chemical Co., Ltd., a polyphenylene ether compound having a styrene structure).

The number average molecular weight (Mn) of the polyphenylene ether compound is preferably 500 to 5000, more preferably 2000 to 5000, and more preferably 2000 to 4000. If the molecular weight is less than 500, the heat resistance of the cured product tends to be insufficient. In addition, if the molecular weight is greater than 5000, the melt viscosity becomes high and sufficient fluidity cannot be obtained, which tends to lead to molding defects. In addition, the reactivity is also reduced, the curing reaction takes a long time, and the amount of unreacted compounds not incorporated into the curing system increases, which leads to a decrease in the glass transition temperature of the cured product and a decrease in the heat resistance of the cured product.

If the number average molecular weight of the polyphenylene ether compound is 500 to 5000, excellent heat resistance and moldability can be achieved while maintaining excellent dielectric properties. The number average molecular weight here can be specifically measured using gel permeation chromatography or the like.

The polyphenylene ether compound may be one obtained by a polymerization reaction, or one obtained by a redistribution reaction of a high molecular weight polyphenylene ether compound having a number average molecular weight of about 10,000 to 30,000. In addition, these may be used as raw materials and reacted with a compound having an ethylenically unsaturated bond, such as methacrylic acid chloride, acrylic acid chloride, or chloromethylstyrene, to impart radical polymerizability. The polyphenylene ether compound obtained by the redistribution reaction is obtained, for example, by heating a high molecular weight polyphenylene ether compound in a solvent such as toluene in the presence of a phenolic compound and a radical initiator to cause a redistribution reaction. The polyphenylene ether compound obtained by the redistribution reaction in this way has hydroxy groups derived from a phenolic compound that contributes to curing at both ends of the molecular chain, and is therefore preferable in that it can maintain even higher heat resistance, and that functional groups can be introduced at both ends of the molecular chain even after modification with a compound having an ethylenically unsaturated bond. In addition, the polyphenylene ether compound obtained by the polymerization reaction is preferable in that it exhibits excellent fluidity.

In the case of a polyphenylene ether compound obtained by a polymerization reaction, the molecular weight of the polyphenylene ether compound can be adjusted by adjusting the polymerization conditions. In the case of a polyphenylene ether compound obtained by a redistribution reaction, the molecular weight of the obtained polyphenylene ether compound can be adjusted by adjusting the conditions of the redistribution reaction. More specifically, it is possible to adjust the amount of the phenolic compound used in the redistribution reaction. That is, the greater the amount of the phenolic compound, the lower the molecular weight of the obtained polyphenylene ether compound. In this case, poly(2,6-dimethyl-1,4-phenylene ether) or the like can be used as a high molecular weight polyphenylene ether compound that undergoes the redistribution reaction. In addition, the phenolic compound used in the redistribution reaction is not particularly limited, but for example, a polyfunctional phenolic compound having two or more phenolic hydroxy groups in the molecule, such as bisphenol A, phenol novolac, cresol novolac, etc., is preferably used. These may be used alone or in combination of two or more.

The content of the polyphenylene ether compound is not particularly limited, but is preferably 5 to 1000 parts by mass, and more preferably 10 to 750 parts by mass, relative to 100 parts by mass of the curable resin composition. When the content of the polyphenylene ether compound is within the above range, it is preferable in that a cured product is obtained that is not only excellent in heat resistance, etc., but also fully exhibits the excellent dielectric properties of the polyphenylene ether compound.

### [Amine Resin]

Amine resin is a compound having two or more amino groups in the molecule. Examples of amine resins include, but are not limited to: diaminodiphenylmethane, diaminodiphenylsulfone, isophoronediamine, naphthalenediamine, aniline novolak (a reaction product of aniline and formalin), N-methylaniline novolak (a reaction product of N-methylaniline and formalin), orthoethylaniline novolak (a reaction product of orthoethylaniline and formalin), a reaction product of 2-methylaniline and formalin, a reaction product of 2,6-diisopropylaniline and formalin, a reaction product of 2,6-diethylaniline and formalin, a reaction product of 2-ethyl-6-ethylaniline and formalin, a reaction product of 2,6-dimethylaniline and formalin, and a reaction product obtained by reacting aniline and xylylene chloride; the aniline resin disclosed in Japanese Patent No. 6429862, a reaction product of aniline and a substituted biphenyl(4,4′-bis(chloromethyl)-1,1′-biphenyl and 4,4′-bis(methoxymethyl)-1,1′-biphenyl, etc.); a reaction product of aniline and substituted phenyls (such as 1,4-bis(chloromethyl)benzene, 1,4-bis(methoxymethyl)benzene, and 1,4-bis(hydroxymethyl)benzene); 4,4′-(1,3-phenylenediisopropylidene)bisaniline; 4,4′-(1,4-phenylenediisopropylidene)bisaniline; a reaction product of aniline and diisopropenylbenzene; dimer diamine, etc. Furthermore, these may be used alone or in combination.

### [Compound Containing an Ethylenically Unsaturated Bond]

The compound containing an ethylenically unsaturated bond is a compound that has one or more ethylenically unsaturated bonds in the molecule that can be polymerized by heat or light, regardless of whether a polymerization initiator is used or not.

Examples of the compound containing an ethylenically unsaturated bond include, but are not limited to: reaction products of the above-mentioned phenolic resin with ethylenically unsaturated bond-containing halogen-based compounds (such as chloromethylstyrene, allyl chloride, methallyl chloride, acrylic acid chloride, and methacrylic acid chloride); reaction products of ethylenically unsaturated bond-containing phenols (such as 2-allylphenol, 2-propenylphenol, 4-allylphenol, 4-propenylphenol, eugenol, and isoeugenol) with halogen-based compounds (such as 1,4-bis(chloromethyl)benzene, 4,4′-bis(chloromethyl) biphenyl, 4,4′-difluorobenzophenone, 4,4′-dichlorobenzophenone, 4,4′-dibromobenzophenone, and cyanuric chloride); reaction products of epoxy resins or alcohols with (meth)acrylic acids (such as acrylic acid, and methacrylic acid); and acid-modified products thereof. In addition, these may be used alone or in combination.

### [Isocyanate Resin]

An isocyanate resin is a compound having two or more isocyanate groups in the molecule. Examples of the isocyanate resin include: aromatic diisocyanates such as p-phenylene diisocyanate, m-phenylene diisocyanate, p-xylylene diisocyanate, m-xylylene diisocyanate, 2,4-tolylene diisocyanate, 2,6-tolylene diisocyanate, 4,4′-diphenylmethane diisocyanate, and naphthalene diisocyanate; aliphatic or alicyclic diisocyanates such as isophorone diisocyanate, hexamethylene diisocyanate, 4,4′-dicyclohexylmethane diisocyanate, hydrogenated xylene diisocyanate, norbornene diisocyanate, and lysine diisocyanate; polyisocyanates such as one or more biuret forms of isocyanate monomers or isocyanate forms obtained by trimerizing the diisocyanate compounds; and polyisocyanates obtained by a urethanization reaction between the isocyanate compounds and polyol compounds, but are not limited thereto. These may be used alone or in combination.

### [Polyamide Resin]

Examples of polyamide resins include reaction products of one or more of diamines, diisocyanates, and oxazolines with dicarboxylic acids, reaction products of diamines with acid chlorides, and ring-opening polymers of lactam compounds. These may be used alone or in combination.

Specific examples of the above-mentioned raw materials are shown below, but are not limited to these.
<Diamines> Ethylenediamine, trimethylenediamine, tetramethylenediamine, pentamethylenediamine, hexamethylenediamine, heptamethylenediamine, octamethylenediamine, nonamethylenediamine, decanediamine, undecanediamine, dodecanediamine, tridecanediamine, tetradecanediamine, pentadecanediamine, hexadecanediamine, heptadecanediamine, octadecanediamine, nonadecanediamine, eicosanediamine, 2-methyl-1,5-diaminopentane, 2-methyl-1,8-diaminooctane, dimer diamine, cyclohexane diamine, bis-(4-aminocyclohexyl) methane, bis(3-methyl-4-aminocyclohexyl) methane, xylylene diamine, norbornane diamine, isophorone diamine, bisaminomethyltricyclodecane, phenylenediamine, diethyltoluenediamine, naphthalenediamine, diaminodiphenylmethane, bis(4-amino-3,5-dimethylphenyl) methane, bis(4-amino-3,5-diethylphenyl) Methane, 4,4′-methylenebis-o-toluidine, 4,4′-methylenebis-o-ethylaniline, 4,4′-methylenebis-2-ethyl-6-methylaniline, 4,4′-methylenebis-2,6-diisopropylaniline, 4,4-ethylenedianiline, diaminodiphenyl sulfone, diaminodiphenyl ether, 1,3-bis(3-aminophenoxy)benzene, 1,3-bis(4-aminophenoxy)benzene, 4,4-bis(4-aminophenoxy) biphenyl, 2,2-bis[4-(4-aminophenoxy)phenyl]propane, bis[4-(4-aminophenoxy)phenyl]sulfone, 2,2-bis(3-amino-4-hydroxyphenyl) propane, 2,2-bis(4-aminophenyl) hexafluoropropane, 4,4′-(1,3-phenylenediisopropylidene)bisaniline, 4,4′-(1,4-phenylenediisopropylidene)bisaniline, 9,9-bis(4-aminophenyl) fluorene, 2,7-diaminofluorene, aminobenzylamine, diaminobenzophenone, and the like.
<Diisocyanates> benzene diisocyanate, toluene diisocyanate, 1,3-bis(isocyanatomethyl)benzene, 1,3-bis(isocyanatomethyl)cyclohexane, bis(4-isocyanatophenyl) methane, isophorone diisocyanate, 1,3-bis(2-isocyanato-2-propyl)benzene, 2,2-bis(4-isocyanatophenyl) hexafluoropropane, dicyclohexylmethane-4,4′-diisocyanate, and the like.
<Dicarboxylic acids> oxalic acid, malonic acid, succinic acid, glutaric acid, adipic acid, suberic acid, azelaic acid, sebacic acid, undecanedioic acid, dodecanedioic acid, terephthalic acid, isophthalic acid, 5-hydroxyisophthalic acid, 2-chloroterephthalic acid, 2-methylterephthalic acid, 5-methylisophthalic acid, 5-sodium sulfoisophthalic acid, hexahydroterephthalic acid, hexahydroisophthalic acid, cyclohexanedicarboxylic acid, biphenyldicarboxylic acid, naphthalenedicarboxylic acid, benzophenonedicarboxylic acid, furandicarboxylic acid, 4,4′-dicarboxydiphenyl ether, 4,4′-dicarboxydiphenyl sulfide, and the like.
<Acid chlorides> acetyl chloride, acrylic acid chloride, methacrylic acid chloride, malonyl chloride, succinic acid dichloride, diglycolyl chloride, glutaric acid dichloride, suberic acid dichloride, sebacic acid dichloride, adipic acid dichloride, dodecandioyl dichloride, azelayl chloride, 2,5-furandicarbonyl dichloride, phthaloyl chloride, isophthaloyl chloride, terephthaloyl chloride, trimesic acid chloride, bis(4-chlorocarbonylphenyl) ether, 4,4′-diphenyldicarbonyl chloride, 4,4′-azodibenzoyl dichloride, etc.
<Lactams> 8-caprolactam, @-undecanelactam, @-laurolactam, etc.

### [Polyimide Resin]

Examples of polyimide resins include, but are not limited to, reaction products of the above diamines with the following tetracarboxylic dianhydrides. These may be used alone or in combination.
<Tetracarboxylic dianhydrides> 4,4′-(hexafluoroisopropylidene)diphthalic anhydride, 5-(2,5-dioxotetrahydro-3-furanyl)-3-methyl-cyclohexene-1,2 dicarboxylic anhydride, pyromellitic dianhydride, 1,2,3,4-benzenetetracarboxylic dianhydride, 3,3′,4,4′-benzophenonetetracarboxylic dianhydride, 2,2′,3,3′-benzophenonetetracarboxylic dianhydride, 3,3′,4,4′-biphenyltetracarboxylic dianhydride, 3,3′,4,4′-diphenylsulfonate tetracarboxylic dianhydride, 2,2′,3,3′-biphenyltetracarboxylic dianhydride, methylene-4,4′-diphthalic dianhydride, 1,1-ethylidene-4,4′-diphthalic dianhydride, 2,2′-propylidene-4,4′-diphthalic dianhydride, 1,2-ethylene-4,4′-diphthalic dianhydride, 1,3-trimethylene-4,4′-diphthalic dianhydride, 1,4-tetramethylene-4,4′-diphthalic dianhydride, 1,5-pentamethylene-4,4′-diphthalic dianhydride, 4,4′-oxydiphthalic dianhydride Anhydride, thio-4,4′-diphthalic dianhydride, sulfonyl-4,4′-diphthalic dianhydride, 1,3-bis(3,4-dicarboxyphenyl)benzene dianhydride, 1,3-bis(3,4-dicarboxyphenoxy)benzene dianhydride, 1,4-bis(3,4-dicarboxyphenoxy)benzene dianhydride, 1,3-bis[2-(3,4-dicarboxyphenyl)-2-propyl]benzene dianhydride, 1,4-bis[2-(3,4-dicarboxyphenyl)-2-propyl]benzene dianhydride, bis[3-(3,4-dicarboxyphenoxy)phenyl]methane dianhydride, bis[4-(3,4-dicarboxyphenoxy)phenyl]methane dianhydride, 2,2-bis[3-(3,4-dicarboxyphenoxy)phenyl]propane dianhydride, 2,2-bis[4-(3,4-dicarboxyphenoxy)phenyl]propane dianhydride, bis(3,4-dicarboxyphenoxy)dimethylsilane dianhydride, 1,3-bis(3,4-dicarboxyphenyl)-1,1,3,3-tetramethyldisiloxane dianhydride, 2,3,6,7-Naphthalene tetracarboxylic dianhydride, 1,4,5,8-naphthalene tetracarboxylic dianhydride, 1,2,5,6-naphthalene tetracarboxylic dianhydride, 3,4,9,10-perylene tetracarboxylic dianhydride, 2,3,6,7-anthracene tetracarboxylic dianhydride, 1,2,7,8-phenanthrene tetracarboxylic dianhydride, ethylene tetracarboxylic dianhydride, 1,2,3,4-butane tetracarboxylic dianhydride, 1,2,3,4-cyclobutane tetracarboxylic dianhydride, cyclopentane tetracarboxylic dianhydride, cyclohexane-1,2,3,4-tetracarboxylic acid dianhydride, cyclohexane-1,2,4,5-tetracarboxylic acid dianhydride, 3,3′,4,4′-bicyclohexyltetracarboxylic acid dianhydride, carbonyl-4,4′-bis(cyclohexane-1,2-dicarboxylic acid)dianhydride, methylene-4,4′-bis(cyclohexane-1,2-dicarboxylic acid)dianhydride, 1,2-ethylene-4,4′-bis(cyclohexane-1,2-dicarboxylic acid)dianhydride, 1,1-ethylidene-4,4′-Bis(cyclohexane-1,2-dicarboxylic acid)dianhydride, 2,2-propylidene-4,4′-bis(cyclohexane-1,2-dicarboxylic acid)dianhydride, oxy-4,4′-bis(cyclohexane-1,2-dicarboxylic acid)dianhydride, thio-4,4′-bis(cyclohexane-1,2-dicarboxylic acid)dianhydride, sulfonyl-4,4′-bis(cyclohexane-1,2-dicarboxylic acid)dianhydride, bicyclo[2,2,2]oct-7-ene-2,3,5,6-tetracarboxylic acid dianhydride, rel-[1S,5R,6R]-3-oxabicyclo[3,2,1]octane-2,4-dione-6-spiro-3′-(tetrahydrofuran-2′,5′-dione), 4-(2,5-dioxotetrahydrofuran-3-yl)-1,2,3,4-tetrahydronaphthalene-1,2-dicarboxylic anhydride, ethylene glycol-bis-(3,4-dicarboxylic anhydride phenyl) ether, 4,4′-biphenyl bis(trimellitic acid monoester acid anhydride), 9,9′-bis(3,4-dicarboxyphenyl) fluorene dianhydride, and the like.

### [Maleimide Compound]

The curable resin composition of the present embodiment may contain a maleimide compound. A maleimide compound is a compound having one or more maleimide groups in the molecule. Examples of the maleimide compound include 4,4′-diphenylmethane bismaleimide, polyphenylmethane maleimide, m-phenylene bismaleimide, 2,2′-bis[4-(4-maleimidophenoxy)phenyl]propane, 3,3′-dimethyl-5,5′-diethyl-4,4′-diphenylmethane bismaleimide, 4-methyl-1,3-phenylene bismaleimide, 4,4′-diphenylether bismaleimide, 4,4′-diphenylsulfone bismaleimide, 1,3-bis(3-maleimidophenoxy)benzene, 1,3-bis(4-maleimidophenoxy)benzene, and Xylox type maleimide compounds (anilix maleimide, manufactured by Mitsui Chemicals Fine Co., Ltd.), biphenylaralkyl-type maleimide compound (solidified by distilling off the solvent under reduced pressure from a resin solution containing the maleimide compound (M2) described in Example 4 of JP 2009-001783 A), bisaminocumylbenzene-type maleimide (maleimide compound described in WO 2020/054601 A), maleimide compounds having an indane structure described in JP 6629692 A or WO 2020/217679 A, the maleimide compounds described in MATERIAL STAGE Vol. 18, No. 12, 2019, “Sequel Epoxy Resin CAS Number Story-Hardener CAS Number Memorandum No. 31 Bismaleimide (1)” and MATERIAL STAGE Vol. 19, No. 2, 2019, “Sequel Epoxy Resin CAS Number Story—Hardener CAS Number Memorandum No. 32 Bismaleimide (2)”. These compounds may be used alone or in combination.

### [Cyanate Ester Resin]

Cyanate ester resin is a cyanate ester compound obtained by reacting a Phenolic resin with a cyanogen halide. Specific examples include dicyanatobenzene, tricyanatobenzene, dicyanatonaphthalene, dicyanatobiphenyl, 2,2′-bis(4-cyanatophenyl) propane, bis(4-cyanatophenyl) methane, bis(3,5-dimethyl-4-cyanatophenyl) methane, 2,2′-bis(3,5-dimethyl-4-cy anatophenyl) propane, 2,2′-bis(4-cyanatophenyl) ethane, 2,2′-bis(4-cy anatophenyl) hexafluoropropane, bis(4-cyanatophenyl) sulfone, bis(4-cy anatophenyl)thioether, phenol novolac cyanate, and phenol-dicyclopentadiene co-condensation product in which the hydroxy groups have been converted to cyanate groups, but are not limited thereto. These may be used alone or in combination.

The cyanate ester compound, the synthesis method of which is described in JP-A-2005-264154, is particularly preferred as the cyanate ester compound, since it has low moisture absorption, flame retardancy, and excellent dielectric properties.

The cyanate ester resin may contain a catalyst such as zinc naphthenate, cobalt naphthenate, copper naphthenate, lead naphthenate, zinc octylate, tin octylate, lead acetylacetonate, or dibutyltin maleate, in order to trimerize the cyanate group to form a sym-triazine ring, as necessary.

The catalyst is preferably used in an amount of 0.0001 to 0.10 parts by mass, and more preferably 0.00015 to 0.0015 parts by mass, based on 100 parts by mass of the cyanate ester resin and the curable resin composition.

### [Polybutadiene and its Modified Products]

Polybutadiene and its modified products are compounds having polybutadiene or a structure derived from polybutadiene in the molecule. The structure derived from polybutadiene may have some or all of the unsaturated bonds converted to single bonds by hydrogenation.

Examples of polybutadiene and its modified products include, but are not limited to, polybutadiene, hydroxy-terminated polybutadiene, (meth)acrylated polybutadiene, carboxylic acid-terminated polybutadiene, amine-terminated polybutadiene, styrene butadiene rubber, etc. Also, these may be used alone or in combination. Of these, polybutadiene or styrene butadiene rubber is preferred from the viewpoint of dielectric properties. Examples of styrene butadiene rubber (SBR) include RICON-100, RICON-181, and RICON-184 (all manufactured by Cray Valley Corporation), and 1,2-SBS (manufactured by Nippon Soda Co., Ltd.). Examples of polybutadiene include B-1000, B-2000, and B-3000 (all manufactured by Nippon Soda Co., Ltd.). The molecular weight of polybutadiene and styrene butadiene rubber is preferably a weight average molecular weight of 500 to 10,000, more preferably 750 to 7,500, and even more preferably 1,000 to 5,000. Below the lower limit of the above range, the amount of volatilization is large, making it difficult to adjust the solid content when preparing the prepreg, and above the upper limit of the above range, the compatibility with other curable resins is deteriorated. In general, in the case of compounds containing heteroatoms such as oxygen and nitrogen, such as bismaleimide and polymaleimide, it is difficult to ensure compatibility with low-polarity compounds such as compounds mainly composed of hydrocarbons or compounds composed only of hydrocarbons due to their polarity. On the other hand, the compound of the present embodiment does not have a skeleton design in which heteroatoms such as oxygen and nitrogen are actively introduced, and therefore has excellent compatibility with materials having low polarity and low dielectric properties and compounds composed only of hydrocarbons.

### [Polystyrene and Modified Products Thereof]

Polystyrene and modified products thereof are polystyrene or compounds having a structure derived from polystyrene in the molecule.

Examples of polystyrene and modified products thereof include polystyrene, styrene-2-isopropenyl-2-oxazoline copolymers (Epocross RPS-1005, RP-61, both manufactured by Nippon Shokubai Co., Ltd.), SEP (styrene-ethylene-propylene copolymer: Septon (registered trademark) 1020, manufactured by Kuraray Co., Ltd.), SEPS (styrene-ethylene-propylene-styrene copolymer: Septon 2002, Septon 2004F, Septon 2005, Septon 2006, Septon 2063, Septon 2104, all manufactured by Kuraray Co., Ltd.), SEEPS (styrene-ethylene/ethylene-propylene-styrene block copolymer: Septon 4003, Septon 4044, Septon 4055, Septon 4077, Septon 4099, all manufactured by Kuraray Co., Ltd.), and SEBS (styrene-ethylene-butylene-styrene block copolymer: Septon 4003, Septon 4044, Septon 4055, Septon 4077, Septon 4099, all manufactured by Kuraray Co., Ltd.), SEEPS-OH (a styrene-ethylene/ethylene propylene-styrene block copolymer having a hydroxy group at the end: SEPTON HG252, manufactured by Kuraray Co., Ltd.), SIS (styrene-isoprene-styrene block copolymer: SEPTON 5125, SEPTON 5127, manufactured by Kuraray Co., Ltd.), hydrogenated SIS (hydrogenated styrene-isoprene-styrene block copolymer: HYBLER (registered trademark) 7125F, HYBLER 7311F, manufactured by Kuraray Co., Ltd.), SIBS (styrene-isobutylene-styrene block copolymer: SIBSTAR073T, SIBSTAR102T, SIBSTAR103T, manufactured by Kaneka Corporation), and SEPTON V9827 (manufactured by Kuraray Co., Ltd.). These may be used alone or in combination. Polystyrene and modified products thereof have higher heat resistance and are less susceptible to oxidative deterioration, so it is preferable that they do not have unsaturated bonds. The weight-average molecular weight of polystyrene and modified products thereof is not particularly limited as long as it is 10,000 or more, but if it is too large, the compatibility with not only polyphenylene ether compounds but also low molecular weight components with a weight-average molecular weight of about 50 to 1,000 and oligomer components with a weight-average molecular weight of about 1,000 to 5,000 deteriorates, making it difficult to ensure mixing and solvent stability, so it is preferably about 10,000 to 300,000.

### [Polyethylene and Modified Products Thereof]

Polyethylene and modified products thereof are compounds having polyethylene or a structure derived from polyethylene in the molecule. Examples of polyethylene and modified products thereof include ethylene-propylene copolymers, ethylene-styrene copolymers, ethylene-propylene-ethylidene norbornene copolymers (EBT: K-8370EM, K-9330M, etc., manufactured by Mitsui Chemicals, Inc.), ethylene-propylene-vinyl norbornene copolymers (VNB-EPT: PX-006M, PX-008M, PX-009M, etc., manufactured by Mitsui Chemicals, Inc.), ethylene-vinyl alcohol copolymers, ethylene-vinyl acetate copolymers, etc., but are not limited thereto. From the viewpoint of improving heat resistance, it is preferable to use ethylene-propylene-ethylidene norbornene copolymers and ethylene-propylene-vinyl norbornene copolymers containing a crosslinkable structure. In addition, these may be used alone or in combination. The weight average molecular weight of the polyethylene and modified products thereof is not particularly limited as long as it is 10,000 or more. However, if it is too large, compatibility with not only the polyphenylene ether compound but also low molecular weight components having a weight average molecular weight of about 50 to 1,000 and oligomer components having a weight average molecular weight of about 1,000 to 5,000 deteriorates, making it difficult to ensure mixing and solvent stability. Therefore, it is preferably about 10,000 to 300,000.

The curable resin composition of the present embodiment can be obtained by preparing the above-mentioned components in a predetermined ratio, pre-curing at 130 to 180° C. for 30 to 500 seconds, and then post-curing at 150 to 200° C. for 2 to 15 hours, whereby a sufficient curing reaction proceeds to obtain a cured product of the present embodiment. Alternatively, the components of the curable resin composition can be uniformly dispersed or dissolved in a solvent or the like, and cured after removing the solvent.

The method for preparing the curable resin composition of the present embodiment is not particularly limited, but each component may be mixed uniformly or may be prepolymerized. For example, a mixture containing the compound of the present embodiment is heated in the presence or absence of a curing accelerator or a polymerization initiator, and in the presence or absence of a solvent to form a prepolymer. Similarly, an amine compound, a compound having an ethylenically unsaturated bond, a maleimide compound, a cyanate ester compound, polybutadiene and its modified products, polystyrene and its modified products, inorganic fillers, and other additives may be added to form a prepolymer. The mixing or prepolymerization of each component is carried out using, for example, an extruder, a kneader, a roll, or the like in the absence of a solvent, and a reaction kettle with a stirrer in the presence of a solvent.

As a method of uniform mixing, the materials are mixed at a temperature in the range of 50 to 100° C. by kneading with a device such as a kneader, roll, or planetary mixer to obtain a uniform resin composition. The obtained resin composition is crushed and then molded into a cylindrical tablet shape using a molding machine such as a tablet machine, or into a granular powder or powder molded body, or these compositions can be melted on a surface support and molded into a sheet shape with a thickness of 0.05 mm to 10 mm to obtain a molded curable resin composition. The obtained molded body is a non-sticky molded body at 0 to 20° C., and even if stored at −25 to 0° C. for one week or more, the flowability and curability are hardly reduced.

The obtained molded body can be molded into a cured product using a transfer molding machine or a compression molding machine.

The curable resin composition of this embodiment can be made into a varnish-like composition (hereinafter, simply referred to as varnish) by adding an organic solvent. The curable resin composition of this embodiment can be dissolved in a solvent such as toluene, xylene, acetone, methyl ethyl ketone, methyl isobutyl ketone, dimethylformamide, dimethylacetamide, N-methylpyrrolidone, etc. as necessary to make a varnish, and impregnated into a substrate such as glass fiber, carbon fiber, polyester fiber, polyamide fiber, alumina fiber, paper, etc., and dried by heating to obtain a prepreg, which can be hot-press molded to obtain a cured product of the curable resin composition of this embodiment. The solvent used in this case is 10 to 70% by weight, preferably 15 to 70% by weight, of the mixture of the curable resin composition of this embodiment and the solvent. In addition, if the composition is in a liquid state, a cured product containing carbon fiber can be obtained as it is, for example, by the RTM method.

The curable resin composition of the present embodiment can also be used as a modifier for a film-type composition. Specifically, it can be used to improve flexibility and the like in the B-stage. Such a film-type resin composition can be obtained as a sheet-like adhesive by applying the curable resin composition of the present embodiment as a varnish onto a release film, removing the solvent under heating, and then performing B-stage formation. This sheet-like adhesive can be used as an interlayer insulating layer in a multilayer substrate or the like.

The curable resin composition of the present embodiment can be heated and melted to reduce the viscosity, and impregnated into reinforced fibers such as glass fibers, carbon fibers, polyester fibers, polyamide fibers, and alumina fibers to obtain a prepreg. Specific examples thereof include glass fibers such as E glass cloth, D glass cloth, S glass cloth, Q glass cloth, spherical glass cloth, NE glass cloth, and T glass cloth, inorganic fibers other than glass, and organic fibers such as polyparaphenylene terephthalamide (Kevlar (registered trademark), manufactured by DuPont), wholly aromatic polyamide, polyester, polyparaphenylene benzoxazole, polyimide, and carbon fibers, but are not particularly limited thereto. The shape of the substrate is not particularly limited, but examples thereof include woven fabric, nonwoven fabric, roving, chopped strand mat, and the like. In addition, as the weaving method of the woven fabric, plain weave, saddle weave, twill weave, and the like are known, and these known weaves can be appropriately selected and used depending on the intended use and performance. In addition, woven fabrics that have been subjected to fiber opening treatment and glass woven fabrics that have been surface-treated with a silane coupling agent or the like are preferably used. The thickness of the substrate is not particularly limited, but is preferably about 0.01 to 0.4 mm. A prepreg can also be obtained by impregnating reinforcing fibers with the varnish and drying the fibers by heating.

Moreover, a laminate can be manufactured using the prepreg. The laminate is not particularly limited as long as it has one or more prepregs, and may have any other layer. The manufacturing method of the laminate can be appropriately applied by a generally known method, and is not particularly limited. For example, when molding a metal foil-clad laminate, a multi-stage press machine, a multi-stage vacuum press machine, a continuous molding machine, an autoclave molding machine, etc. can be used, and the prepregs are laminated together and heated and pressurized to obtain a laminate. At this time, the heating temperature is not particularly limited, but is preferably 65 to 300° C., and more preferably 120 to 270° C. In addition, the pressure to be applied is not particularly limited, but if the pressure is too high, it is difficult to adjust the solid content of the resin of the laminate, and the quality is not stable, and if the pressure is too low, air bubbles and adhesion between the laminates are deteriorated, so that 2.0 to 5.0 MPa is preferable, and 2.5 to 4.0 MPa is more preferable. The laminate of this embodiment can be suitably used as a metal foil-clad laminate described later by providing a layer made of metal foil.

The prepreg is cut into a desired shape and laminated with copper foil or the like as necessary. The laminate is then heated and cured while applying pressure thereto by press molding, autoclave molding, sheet winding molding or the like, to obtain an electrical and electronic laminate (printed wiring board) or a carbon fiber reinforced material.

The curable resin composition of this embodiment can also be made into a resin sheet. As a method for obtaining a resin sheet from the curable resin composition of this embodiment, for example, a method of applying the curable resin composition onto a support film (support), drying it, and forming a resin composition layer on the support film can be mentioned. When the curable resin composition of this embodiment is used for a resin sheet, it is essential that the film softens under the temperature conditions (70° C. to 140° C.) of lamination in the vacuum lamination method, and simultaneously with lamination of the circuit board, exhibits a fluidity (resin flow) that allows resin filling in via holes or through holes present in the circuit board, and it is preferable to blend each of the above components so as to express such characteristics. In addition, in the obtained resin sheet or circuit board (copper-clad laminate, etc.), a phenomenon that locally different characteristic values are exhibited due to phase separation or the like is not caused, and a certain performance is expressed at any part, so that a uniform appearance is required.

Here, the diameter of the through-holes in the circuit board is 0.1 to 0.5 mm, and the depth is 0.1 to 1.2 mm, and it is preferable to make it possible to fill the resin within this range. When laminating both sides of the circuit board, it is preferable to fill about ½ of the through-holes.

A specific method for producing the above-mentioned resin sheet includes a method in which an organic solvent is blended with a resin composition to prepare a varnish, the resin composition in the form of the varnish is applied to the surface of a support film (Y), and the organic solvent is dried by heating or blowing hot air, etc., to form a resin composition layer (X).

As the organic solvent to be used, for example, ketones such as acetone, methyl ethyl ketone, cyclohexanone, etc., acetate esters such as ethyl acetate, butyl acetate, cellosolve acetate, propylene glycol monomethyl ether acetate, carbitol acetate, etc., carbitols such as cellosolve, butyl carbitol, etc., aromatic hydrocarbons such as toluene, xylene, etc., dimethylformamide, dimethylacetamide, N-methylpyrrolidone, etc. are preferably used. It is preferable to use the organic solvent in such a proportion that the nonvolatile content is 30 to 60 mass % of the total.

The thickness of the resin composition layer (X) formed must be equal to or greater than the thickness of the conductor layer of the circuit board to which the resin composition layer (X) is laminated. Since the thickness of the conductor layer of the circuit board is in the range of 5 to 70 μm, the thickness of the resin composition layer (X) is preferably 10 to 100 μm. The resin composition layer (X) in this embodiment may be protected with a protective film described below. By protecting the resin composition layer (X) with a protective film, it is possible to prevent the adhesion of dirt and the like to the surface of the resin composition layer (X) and scratches.

Examples of the support film and protective film include polyolefins such as polyethylene, polypropylene, and polyvinyl chloride, polyesters such as polyethylene terephthalate (PET) and polyethylene naphthalate, polycarbonate, polyimide, and even release paper and metal foils such as copper foil and aluminum foil. The support film and protective film may be subjected to a matte treatment, a corona treatment, or a release treatment. The thickness of the support film is not particularly limited, but is in the range of 10 to 150 μm, and preferably 25 to 50 μm. The thickness of the protective film is preferably 1 to 40 μm.

The support film (Y) is peeled off after laminating the resin composition layer (X) on a circuit board, or after forming an insulating layer by heat-curing the resin composition layer (X). If the support film (Y) is peeled off after the resin composition layer (X) constituting the resin sheet is heat-cured, adhesion of dust and the like during the curing process can be prevented. When peeling off after the resin composition layer (X) is cured, the support film (Y) is previously subjected to a release treatment.

A multilayer printed circuit board can be manufactured from the resin sheet obtained as described above. For example, when the resin composition layer (X) is protected by a protective film, the protective film is peeled off, and then the resin composition layer (X) is laminated on one or both sides of the circuit board so as to be in direct contact with the circuit board, for example, by a vacuum lamination method. The lamination method may be a batch type or a continuous type using a roll. If necessary, the resin sheet and the circuit board may be heated (preheated) before lamination. The lamination conditions are preferably a pressure bonding temperature (lamination temperature) of 70 to 140° C., a pressure bonding pressure of 1 to 11 kgf/cm² (9.8×10⁴ to 107.9×10⁴ N/m²), and lamination is preferably performed under reduced pressure of 20 mmHg (26.7 hPa) or less.

In addition, the curable resin composition of the present embodiment can be used to manufacture a semiconductor device, such as a dual in-line package (DIP), a quad flat package (QFP), a ball grid array (BGA), a chip size package (CSP), a small outline package (SOP), a thin small outline package (TSOP), a thin quad flat package (TQFP), etc.

The curable resin composition of the present embodiment and its cured product can be used in a wide range of fields. Specifically, it can be used in various applications such as molding materials, adhesives, composite materials, and paints. The cured product of the curable resin composition described in this embodiment exhibits excellent heat resistance and dielectric properties, and is therefore suitable for use in electrical and electronic components such as semiconductor element encapsulants, liquid crystal display element encapsulants, organic EL element encapsulants, laminates (printed wiring boards, BGA substrates, build-up substrates, etc.); high-strength structural composite materials such as carbon fiber reinforced plastics and glass fiber reinforced plastics; and other lightweight and, 3D printing, and the like.

### EXAMPLES

The present invention will now be described in more detail with reference to examples. Unless otherwise specified, all parts are by weight. However, the present embodiment is not limited to these examples.

The various analytical methods used in the examples are described below.

### <High Performance Liquid Chromatography (HP-LC)>

HP-LC: Liquid delivery unit (LC-20AB), online degasser (DGU-20A3), autosampler (SIL-20A), column oven (CTO-20A), system controller (CBM-20A) (all manufactured by Shimadzu Corporation)

Column: ODS-2 (manufactured by GL Sciences)

Eluent: tetrahydrofuran:water=3:1 (no gradient)

Flow rate: 0.5 ml/min.

Column temperature: 40° C.

Detection: photodiode array detector (SPD-M20A) (manufactured by Shimadzu Corporation)

### Example 1

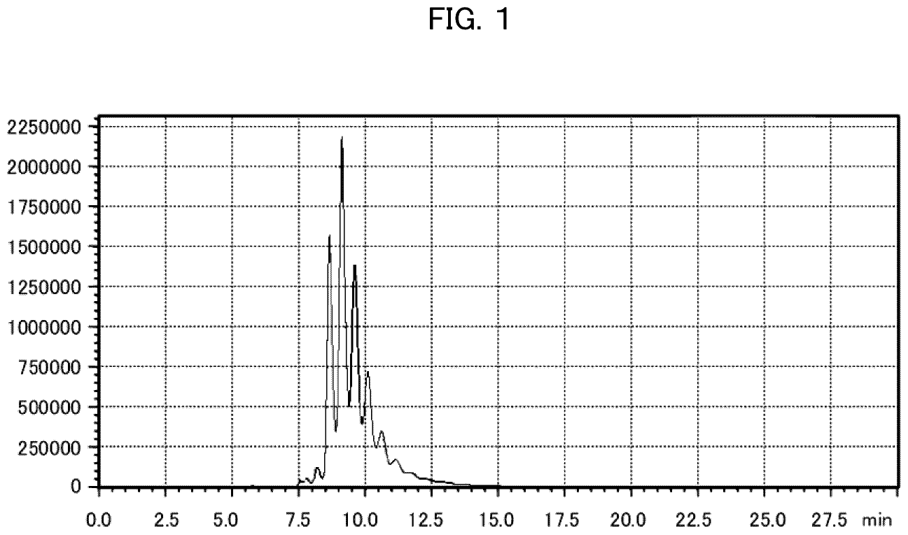

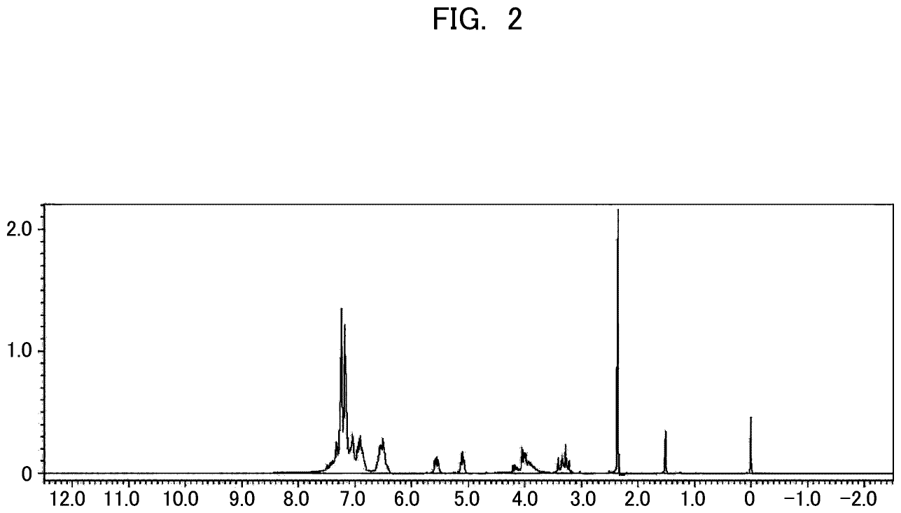

While purging with nitrogen into a flask equipped with a thermometer, a cooling tube, a stirrer, and a Dean-Stark tube, 16.6 parts of fluorene, 32.4 parts of benzyl alcohol, and 2.45 parts of methanesulfonic acid were added, and the temperature was raised to 130° C. The reaction was carried out for 6 hours while removing the generated water from the system using a Dean-Stark tube. After cooling to 80° C., 1.1 parts of sodium hydroxide were added and stirred for 30 minutes. After cooling the internal temperature to 60° C., 100 parts of dimethyl sulfoxide (hereinafter also referred to as DMSO) and 12 parts of sodium hydroxide were added and stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene-95:5 (molar ratio), purity 96.87% by weight) was added dropwise over 2 hours, and the mixture was allowed to react at 65° C. for 4 hours. 200 parts of toluene was added, and the organic layer was washed four times with 100 parts of water. The obtained organic layer was concentrated to obtain 59.1 parts of compound (F1) represented by the following formula (f-1). The HP-LC chart of the obtained compound (F1) is shown in FIG. 1. The ¹H-NMR data (deuterated chloroform) of the obtained compound is shown in FIG. 2. A signal derived from the terminal hydrogen of the vinyl group (two hydrogens per vinyl group) was observed at 5.05-5.65 ppm in the ¹H-NMR chart, and a signal derived from the methylene bond of the benzyl group modified with benzyl alcohol was observed at 2.36 ppm. The integral value of the peak derived from the terminal hydrogen of the vinyl group was 4.00, and the integral value of the peak derived from the methylene bond of the benzyl group modified with benzyl alcohol was 6.04. The average mₐᵥₑ of the values of m in the following formula (f-1) calculated from these values was 1.51.

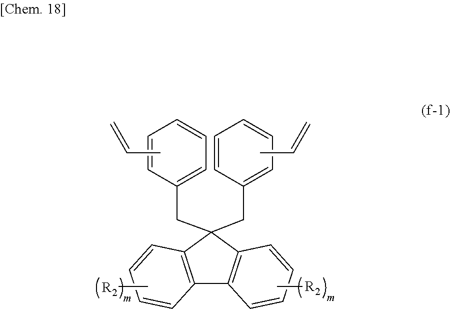

In the above formula (f-1), each of the multiple R₂ independently represents a hydrocarbon group represented by the following formula (f-a-1):

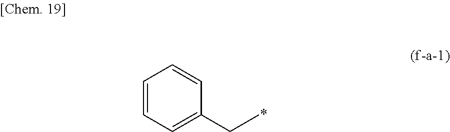

(In the above formula (f-a-1), * represents the bonding position to the fluorene structure of formula (f-1).)

### Example 2

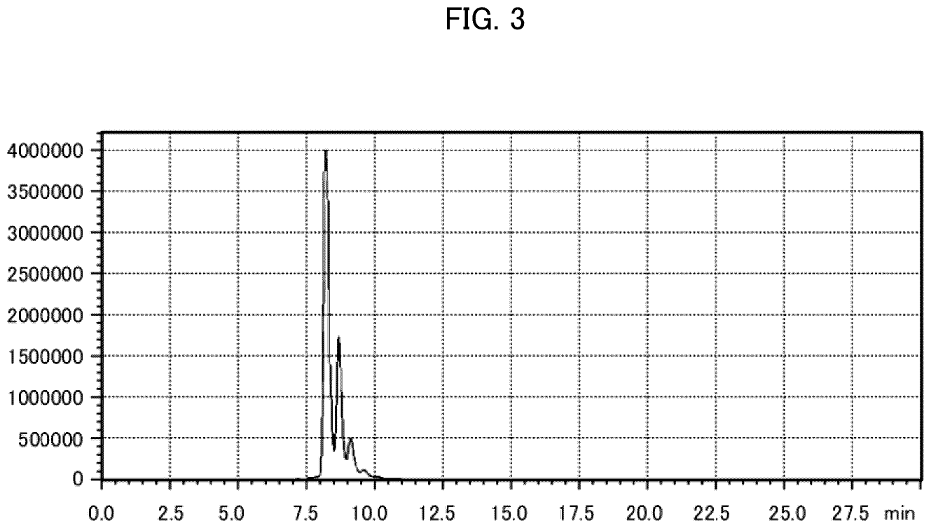

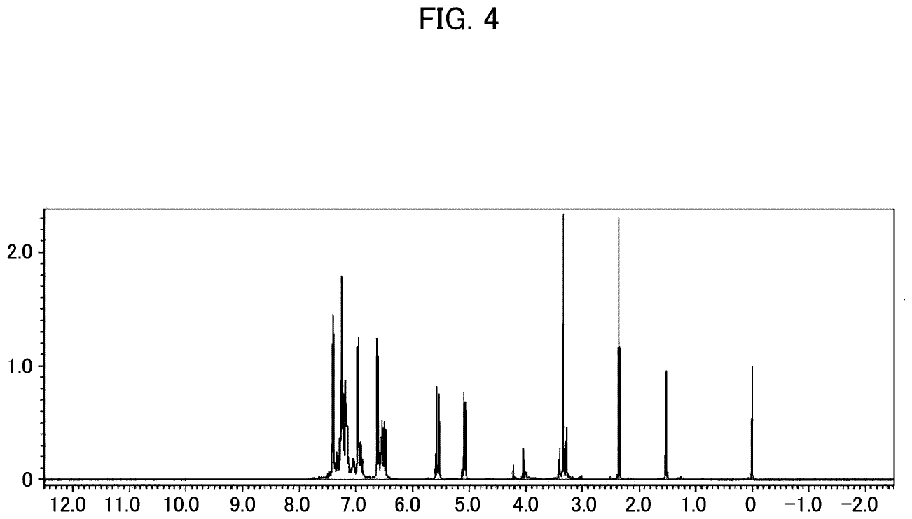

While nitrogen purging was applied to a flask equipped with a thermometer, a condenser, a stirrer, and a Dean-Stark tube, 50 parts of methylcyclohexane, 33.3 parts of fluorene, 10.8 parts of benzyl alcohol, and 2.2 parts of methanesulfonic acid were added, and the temperature was raised to 107° C. The reaction was carried out for 3 hours while removing the generated water from the system using a Dean-Stark tube. After cooling to 80° C., 1.0 part of sodium hydroxide was added and stirred for 30 minutes. After cooling the internal temperature to 60° C., 200 parts of dimethylsulfoxide and 24 parts of sodium hydroxide were added and stirred at 60° C. for 30 minutes. Thereafter, while maintaining the internal temperature at 65° C. or less, 58.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 0.5 hours, and the mixture was allowed to react at 65° C. for 4 hours. 200 parts of toluene was added, and the organic layer was washed four times with 100 parts of water. The organic layer obtained was concentrated to obtain 90.5 parts of compound (F2) represented by formula (f-1). The HP-LC chart of the obtained compound (F2) is shown in FIG. 3. The ¹H-NMR data (deuterated chloroform) of the obtained compound is shown in FIG. 4. A signal derived from the terminal hydrogen of the vinyl group (two hydrogens per vinyl group) was observed at 5.05-5.65 ppm in the ¹H-NMR chart, and a signal derived from the methylene bond of the benzyl group modified with benzyl alcohol was observed at 2.36 ppm. The integral value of the peak derived from the terminal hydrogen of the vinyl group was 4.00, and the integral value of the peak derived from the methylene bond of the benzyl group modified with benzyl alcohol was 2.22. The average mₐᵥₑ of the values of m in the formula (f-1) calculated from these values was 0.56.

### Example 3

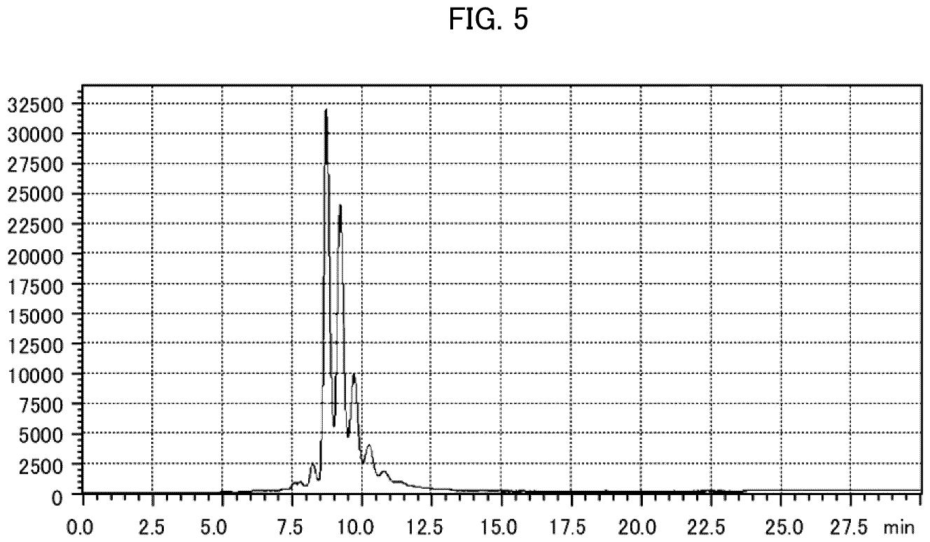

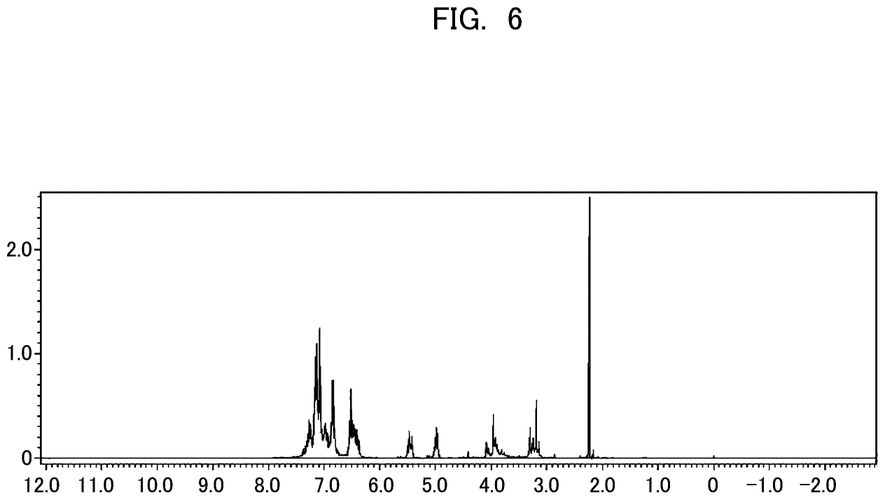

While purging nitrogen into a flask equipped with a thermometer, a cooling tube, a stirrer, and a Dean-Stark tube, 16.6 parts of fluorene, 9.1 parts of methylcyclohexane, and 2.8 parts of methanesulfonic acid were added, and the internal temperature was raised to 95° C. While maintaining the internal temperature at 95-100° C., 10.8 parts of benzyl alcohol were added dropwise over 1 hour, and the reaction was carried out at 98° C. for 3 hours while removing the generated water from the system using a Dean-Stark tube. Next, while maintaining the internal temperature at 95-100° C., 10.8 parts of benzyl alcohol were added dropwise over 1 hour, and the reaction was carried out at 98° C. for 6 hours while removing the generated water from the system using a Dean-Stark tube. After cooling the internal temperature to 80° C., 1.3 parts of sodium hydroxide were added, 100 parts of dimethyl sulfoxide, and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 30 minutes, and the reaction was carried out at 65° C. for 2 hours. 150 parts of toluene was added, and the organic layer was washed five times with 100 parts of water. The obtained organic layer was concentrated to obtain 51.4 parts of compound (F3) represented by formula (f-1). The HP-LC chart of the obtained compound (F3) is shown in FIG. 5. The ¹H-NMR data (deuterated chloroform) of the obtained compound is shown in FIG. 6. A signal derived from the terminal hydrogen of the vinyl group (two hydrogens per vinyl group) was observed at 4.92-5.53 ppm in the ¹H-NMR chart, and a signal derived from the methylene bond of the benzyl group modified with benzyl alcohol was observed at 3.60-4.20 ppm. The integral value of the peak derived from the terminal hydrogen of the vinyl group was 4.08, and the integral value of the peak derived from the methylene bond of the benzyl group modified with benzyl alcohol was 3.80. The average of the values m in the formula (f-1) calculated from these values was 1.02.

### Example 4

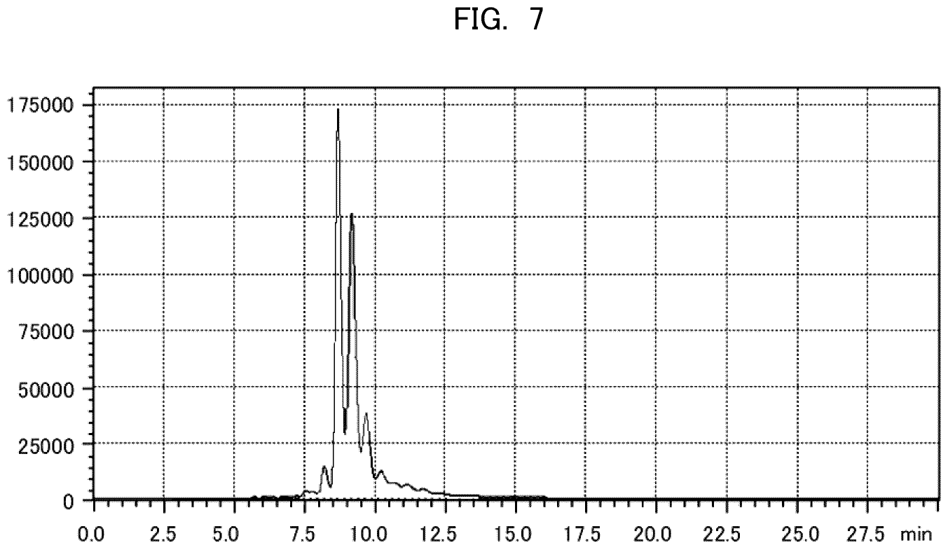

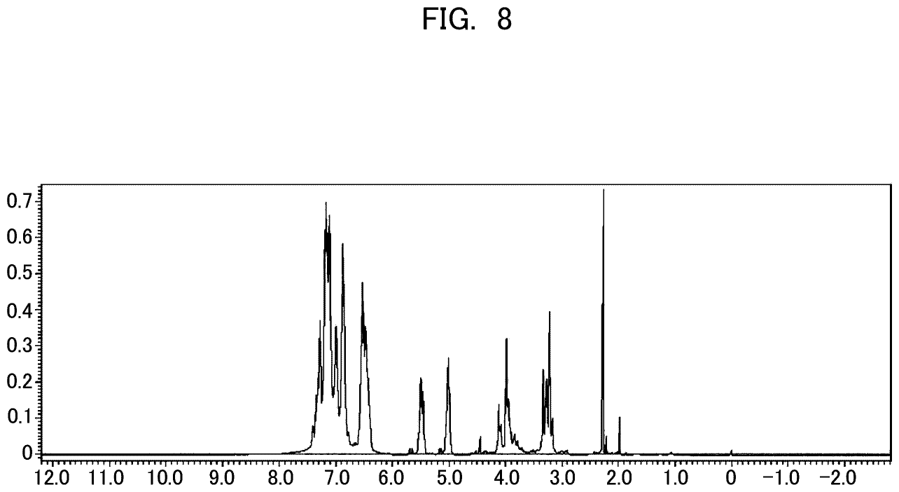

While purging with nitrogen into a flask equipped with a thermometer, a cooling tube, a stirrer, and a Dean-Stark tube, 16.6 parts of fluorene, 9.1 parts of methylcyclohexane, and 2.8 parts of methanesulfonic acid were added, and the internal temperature was raised to 95° C. While maintaining the internal temperature at 95-100° C., 10.8 parts of benzyl alcohol were added dropwise over 1 hour, and the reaction was carried out at 98° C. for 3 hours while removing the generated water from the system using a Dean-Stark. Next, while maintaining the internal temperature at 95-100° C., 5.4 parts of benzyl alcohol were added dropwise over 30 minutes, and the reaction was carried out at 98° C. for 3 hours while removing the generated water from the system using a Dean-Stark. Furthermore, while maintaining the internal temperature at 95-100° C., 2.7 parts of benzyl alcohol were added dropwise over 15 minutes, and the reaction was carried out at 98° C. for 2 hours while removing the generated water from the system using a Dean-Stark. After cooling the internal temperature to 80° C., 1.3 parts of sodium hydroxide was added, 100 parts of dimethyl sulfoxide, and 12 parts of sodium hydroxide were added, and the mixture was stirred at 60° C. for 30 minutes. Thereafter, while maintaining the internal temperature at 65° C. or less, 29.0 parts of CMS-14 (manufactured by AGC Seimi Chemical Co., Ltd., a mixture of 4-chloromethylstyrene and 3-chloromethylstyrene, 4-chloromethylstyrene: 3-chloromethylstyrene=95:5 (molar ratio), purity 96.87 wt %) was added dropwise over 1 hour, and the mixture was reacted at 65° C. for 2 hours. 150 parts of toluene was added, and the organic layer was washed five times with 100 parts of water. The obtained organic layer was concentrated to obtain 29.4 parts of compound (F4) represented by the formula (f-1). The HP-LC chart of the obtained compound (F4) is shown in FIG. 7. Also, the ¹H-NMR data (heavy chloroform) of the obtained compound is shown in FIG. 8. A signal derived from the terminal hydrogen of the vinyl group (two hydrogens per vinyl group) was observed at 5.03-5.62 ppm in the ¹H-NMR chart, and a signal derived from the methylene bond of the benzyl group modified with benzyl alcohol was observed at 3.60-4.20 ppm. The integral value of the peak derived from the terminal hydrogen of the vinyl group was 4.00, and the integral value of the peak derived from the methylene bond of the benzyl group modified with benzyl alcohol was 3.34. The average mₐᵥₑ of the values of m in the formula (f-1) calculated from these values was 0.84.

### Example 5

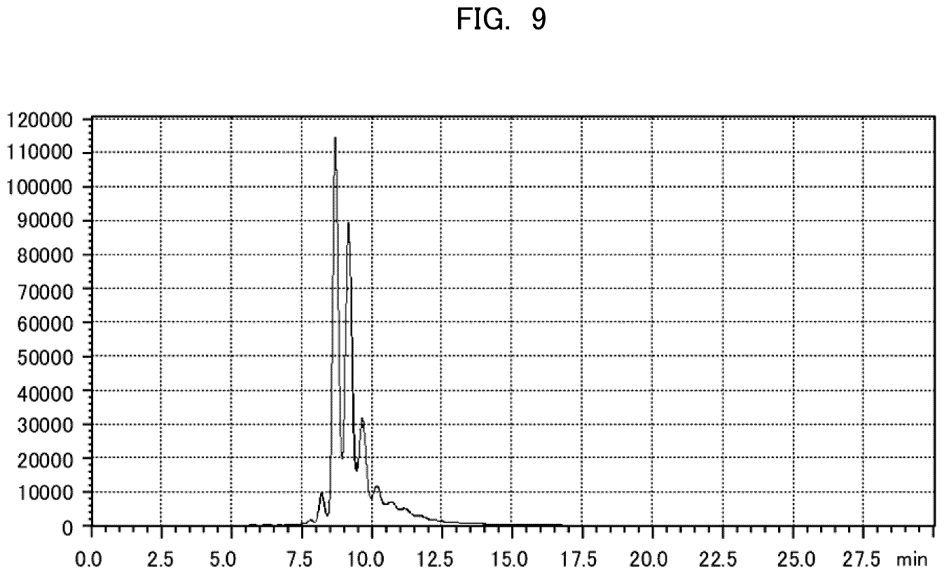

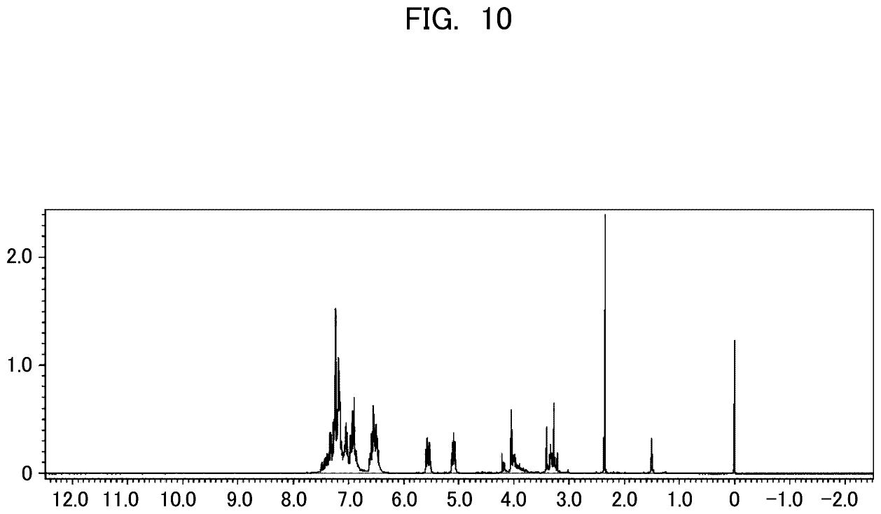

While purging nitrogen into a flask equipped with a thermometer, a cooling tube, a stirrer, and a Dean-Stark tube, 16.6 parts of fluorene, 54.8 parts of methylcyclohexane, and 2.8 parts of methanesulfonic acid were added, and the internal temperature was raised to 95° C. While maintaining the internal temperature at 95-100° C., 10.8 parts of benzyl alcohol were added dropwise over 1 hour, and the reaction was carried out at 98° C. for 6 hours while removing the generated water from the system using a Dean-Stark tube. Subsequently, while maintaining the internal temperature at 95-100° C., 10.8 parts of benzyl alcohol were added dropwise over 1 hour, and the reaction was carried out at 98° C. for 5 hours while removing the generated water from the system using a Dean-Stark tube. After cooling the internal temperature to 80° C., 2.0 parts of sodium hydroxide were added. Then, 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 2 hours, and the mixture was allowed to react at 65° C. for 3 hours. 150 parts of toluene was added, and the organic layer was washed five times with 100 parts of water. The obtained organic layer was concentrated to obtain 43.8 parts of compound (F5) represented by formula (f-1). The HP-LC chart of the obtained compound (F5) is shown in FIG. 9. The ¹H-NMR data (deuterated chloroform) of the obtained compound is shown in FIG. 10. A signal derived from the terminal hydrogen of the vinyl group (two hydrogens per vinyl group) was observed at 5.03-5.62 ppm in the ¹H-NMR chart, and a signal derived from the methylene bond of the benzyl group modified with benzyl alcohol was observed at 3.60-4.20 ppm. The integral value of the peak derived from the terminal hydrogen of the vinyl group was 4.00, and the integral value of the peak derived from the methylene bond of the benzyl group modified with benzyl alcohol was 3.98. The average mₐᵥₑ of the values of m in the formula (f-1) calculated from these values was 0.99.

### Example 6

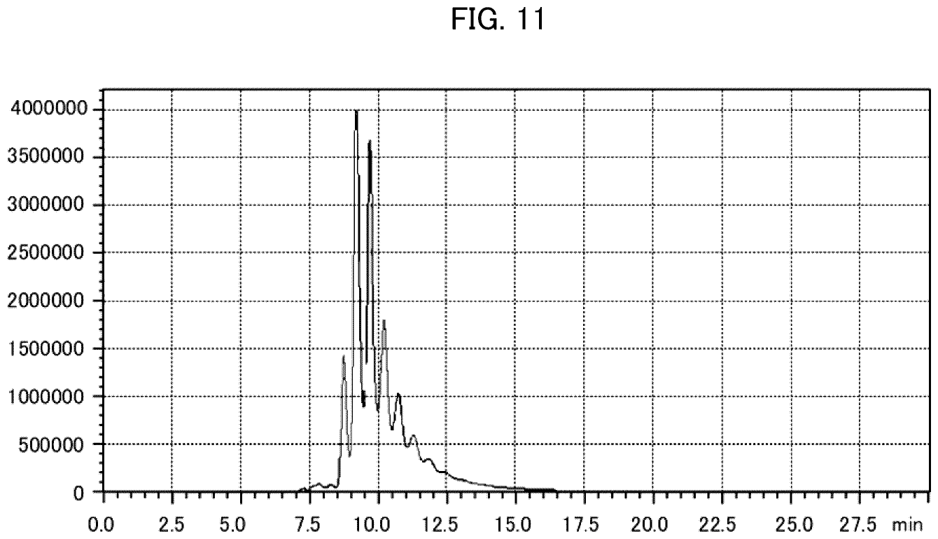

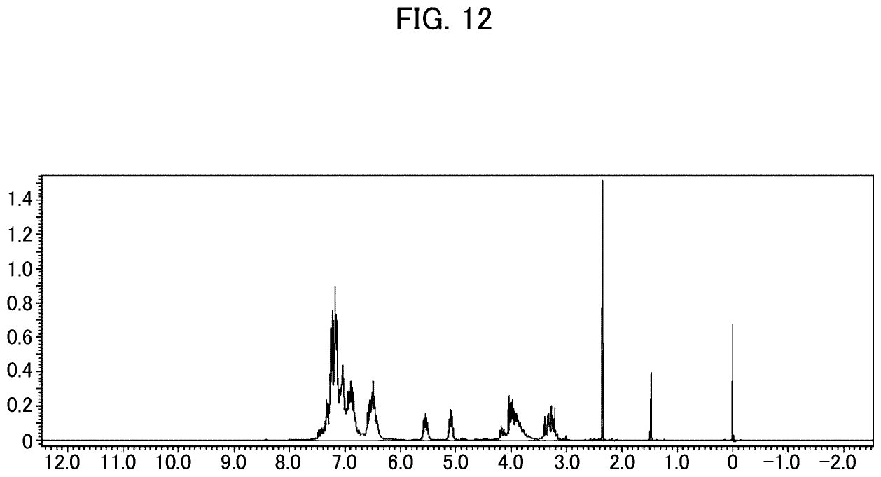

While nitrogen purging was applied to a flask equipped with a thermometer, a cooling tube, a stirrer, and a Dean-Stark tube, 16.6 parts of fluorene, 43.3 parts of benzyl alcohol, and 3.0 parts of methanesulfonic acid were added, and the internal temperature was raised to 130° C. The reaction was carried out for 4 hours at 130° C. while removing the generated water from the system using a Dean-Stark tube. After cooling the internal temperature to 80° C., 1.4 parts of sodium hydroxide were added, 100 parts of dimethyl sulfoxide, and 12 parts of sodium hydroxide were added, and the mixture was stirred at 60° C. for 30 minutes. Thereafter, while maintaining the internal temperature at 65° C. or less, 29.0 parts of CMS-14 (manufactured by AGC Seimi Chemical Co., Ltd., a mixture of 4-chloromethylstyrene and 3-chloromethylstyrene, 4-chloromethylstyrene: 3-chloromethylstyrene=95:5 (mol ratio), purity 96.87 wt %) was added dropwise over 1 hour, and the mixture was reacted at 65° C. for 3 hours. 200 parts of toluene was added, and the organic layer was washed five times with 100 parts of water. The obtained organic layer was concentrated to obtain 71.3 parts of compound (F6) represented by the formula (f-1). The HP-LC chart of the obtained compound (F6) is shown in FIG. 11. Also, ¹H-NMR data (deuterated chloroform) of the obtained compound is shown in FIG. 12. A signal derived from the terminal hydrogen of the vinyl group (two hydrogens per vinyl group) was observed at 5.03-5.62 ppm in the ¹H-NMR chart, and a signal derived from the methylene bond of the benzyl group modified with benzyl alcohol was observed at 3.60-4.20 ppm. The integral value of the peak derived from the terminal hydrogen of the vinyl group was 4.00, and the integral value of the peak derived from the methylene bond of the benzyl group modified with benzyl alcohol was 8.02. The average mₐᵥₑ of the values of m in the formula (f-1) calculated from this was 2.01.

### Example 7

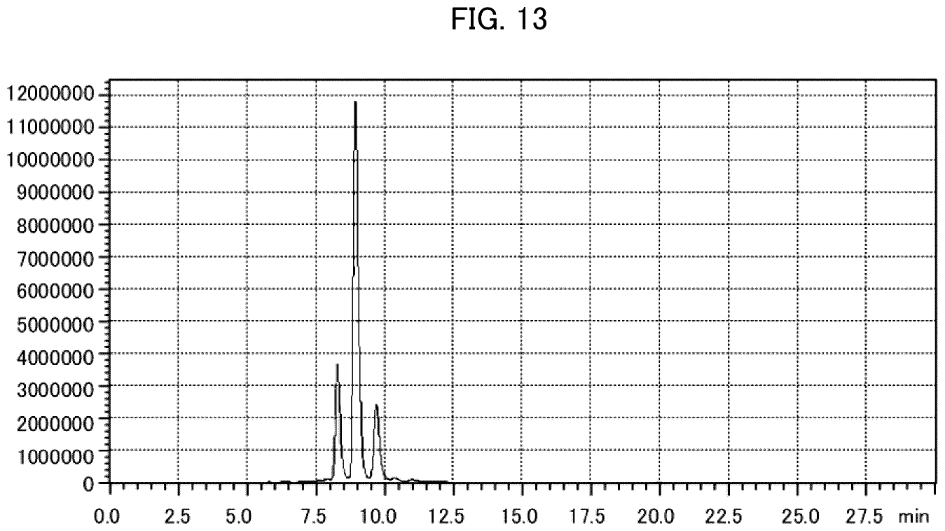

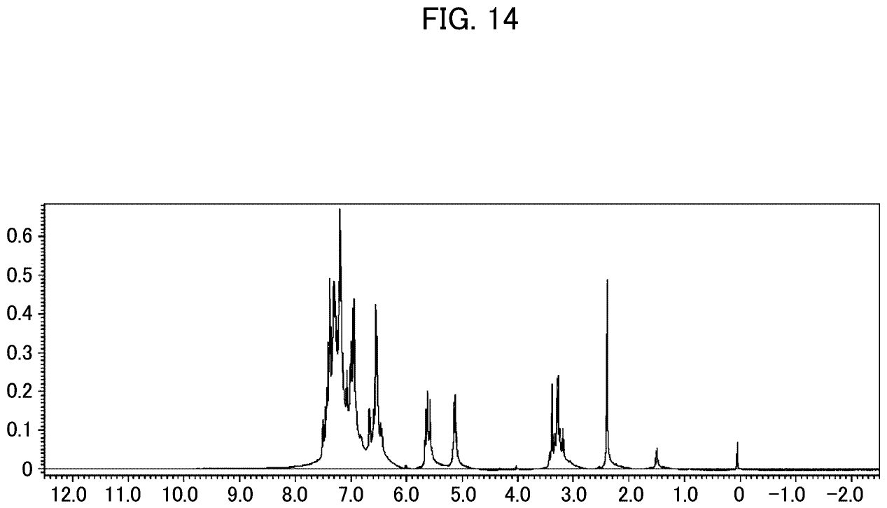

While nitrogen purging was applied to a flask equipped with a thermometer, a cooling tube, a stirrer, and a Dean-Stark tube, 16.6 parts of fluorene, 18.4 parts of benzhydrol, 70 parts of methylcyclohexane, and 1.8 parts of methanesulfonic acid were added, and the temperature was raised to 100° C., and the reaction was carried out for 6 hours while removing the generated water from the system using a Dean-Stark tube by azeotropic dehydration. 2.6 g of 30% aqueous sodium hydroxide was added, and methylcyclohexane was collected until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 2 hours, and the mixture was allowed to react at 65° C. for 3 hours. 150 parts of toluene was added, and the organic layer was washed five times with 100 parts of water. The obtained organic layer was concentrated to obtain 50 parts of compound (F7) represented by the following formula (f-2). The HP-LC chart of the obtained compound (F7) is shown in FIG. 13. The ¹H-NMR data (deuterated chloroform) of the obtained compound is shown in FIG. 14. A signal derived from the terminal hydrogen of the vinyl group (two hydrogens per vinyl group) was observed at 4.80-5.80 ppm in the ¹H-NMR chart, and a signal derived from the structure modified with benzhydrol was observed at 2.80-3.30 ppm. The integral value of the peak derived from the terminal hydrogen of the vinyl group was 4.00, and the integral value of the peak derived from each hydrogen of the methine group modified with benzhydrol was 1.04. The average mₐᵥₑ of the values m in the following formula (f-2) calculated from these values was 0.52.

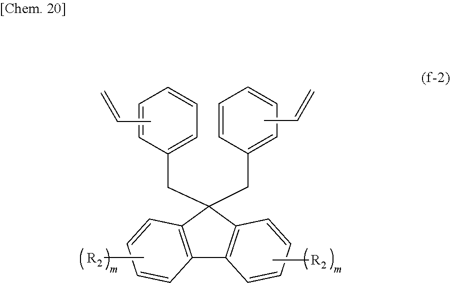

In the above formula (f-2), a plurality of R₂ each independently represent a hydrocarbon group represented by the following formula (f-a-2).

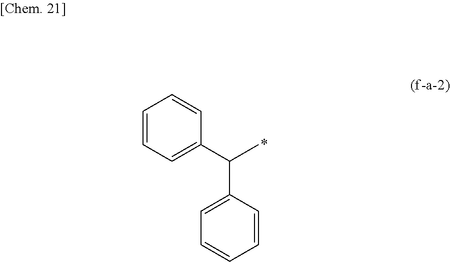

In the above formula (f-a-2), * represents the bonding position to the fluorene structure of formula (f-2).

### Example 8

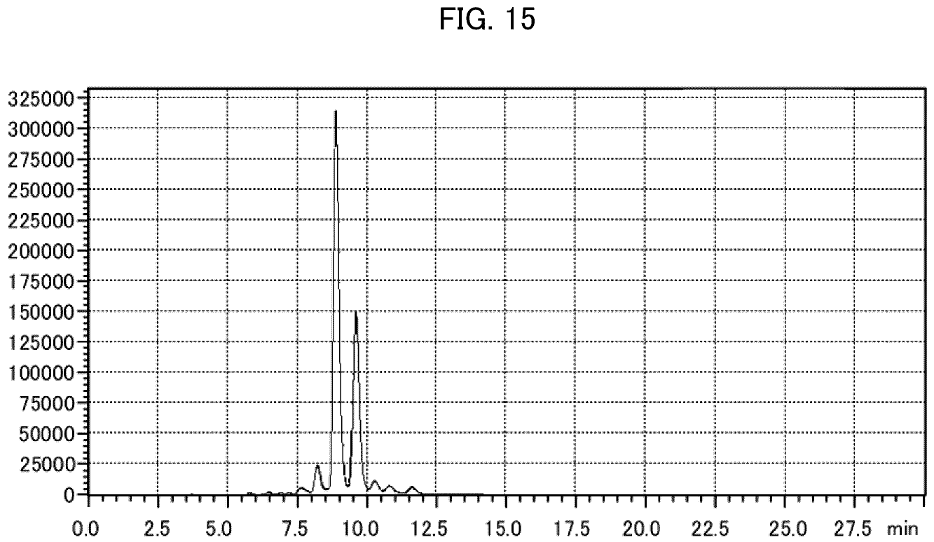

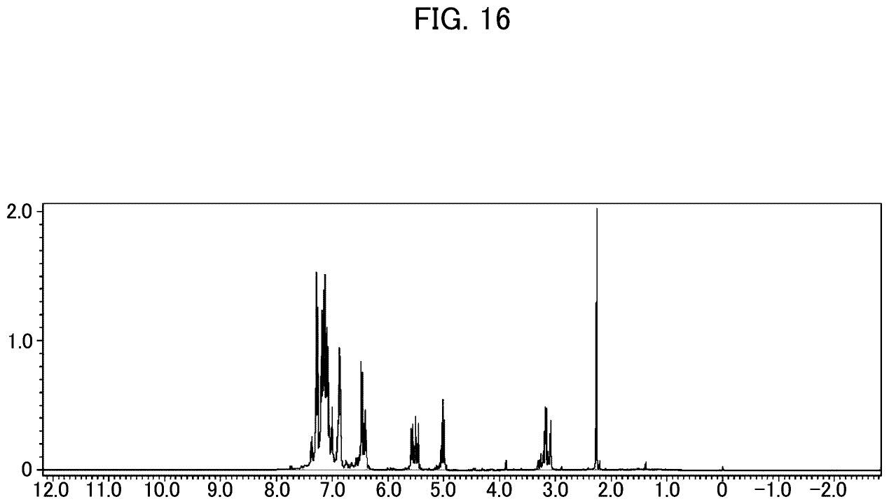

While nitrogen purging was applied to a flask equipped with a thermometer, a cooling tube, a stirrer, and a Dean-Stark tube, 16.6 parts of fluorene, 18.4 parts of benzhydrol, 70 parts of methylcyclohexane, and 1.8 parts of methanesulfonic acid were added, and the temperature was raised to 100° C., and the reaction was carried out for 4 hours while removing the generated water from the system by azeotropic dehydration using a Dean-Stark tube. After cooling the internal temperature to 60° C., 9.2 parts of benzhydrol were added, and the temperature was raised to 100° C., and the reaction was carried out for 3 hours while removing the generated water from the system by azeotropic dehydration using a Dean-Stark tube. After cooling the internal temperature to 60° C., 2.8 parts of 30% aqueous sodium hydroxide solution were added, and methylcyclohexane was collected until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 30 minutes, and the mixture was allowed to react at 65° C. for 4 hours. 150 parts of toluene was added, and the organic layer was washed five times with 100 parts of water. The obtained organic layer was concentrated to obtain 62.3 parts of compound (F8) represented by formula (f-2) above. The HP-LC chart of the obtained compound (F8) is shown in FIG. 15. In addition, the ¹H-NMR data (deuterated chloroform) of the obtained compound is shown in FIG. 16. A signal derived from the terminal hydrogen of the vinyl group (two hydrogens per vinyl group) was observed at 4.80-5.80 ppm in the 1H-NMR chart, and a signal derived from the structure modified with benzhydrol was observed at 2.80-3.30 ppm. The integral value of the peak derived from the terminal hydrogen of the vinyl group was 4.00, and the integral value of the peak derived from each hydrogen of the methine group modified with benzhydrol was 2.75. The average mₐᵥₑ of the values m in the above formula (f-2) calculated from these values was 1.38.

### Example 9

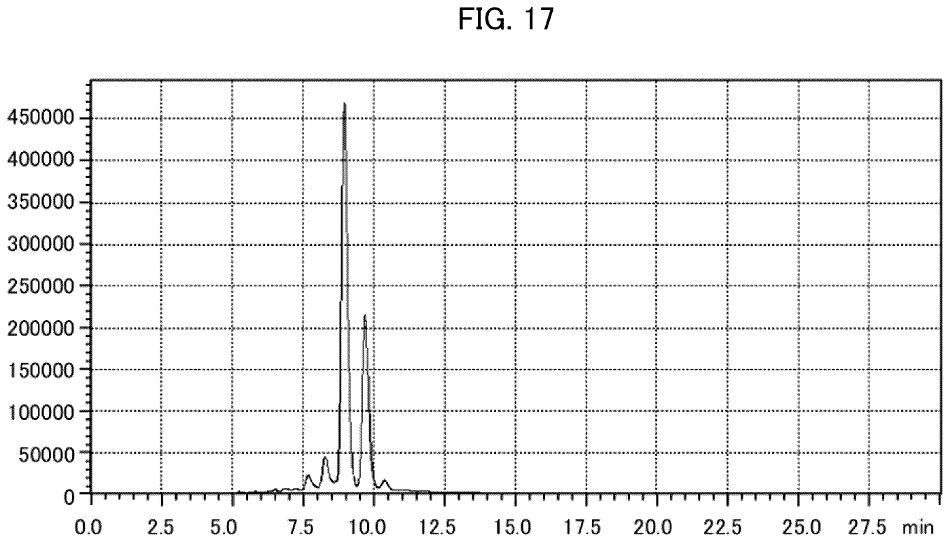

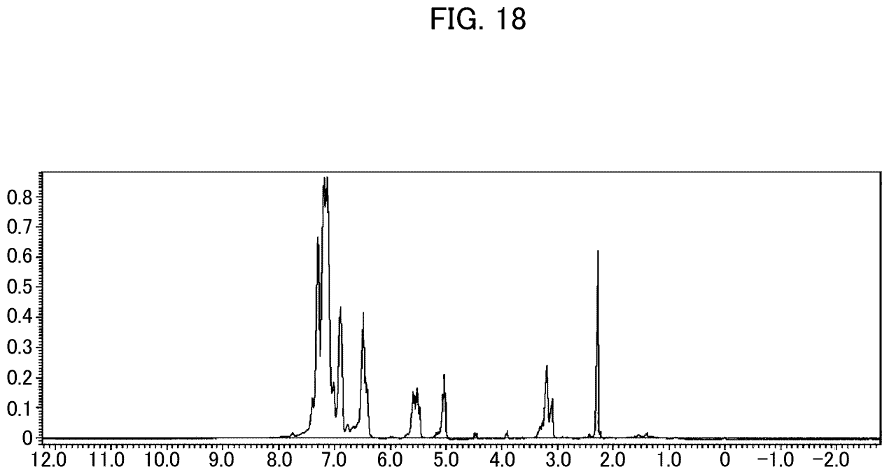

While nitrogen purging was applied to a flask equipped with a thermometer, a cooling tube, a stirrer, and a Dean-Stark tube, 16.6 parts of fluorene, 18.4 parts of benzhydrol, 70 parts of methylcyclohexane, and 2.2 parts of methanesulfonic acid were added, and the temperature was raised to 98° C., and the reaction was carried out for 4 hours while removing the generated water from the system using a Dean-Stark tube by azeotropic dehydration. After cooling the internal temperature to 60° C., 9.2 parts of benzhydrol were added, and the temperature was raised to 98° C., and the reaction was carried out for 3 hours while removing the generated water from the system using a Dean-Stark tube by azeotropic dehydration. After cooling the internal temperature to 60° C., 3.4 parts of 30% sodium hydroxide aqueous solution were added, and methylcyclohexane was collected until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 30.0 parts of CMS-14 (AGC Seimi Chemical Co., Ltd., mixture of 4-chloromethylstyrene and 3-chloromethylstyrene, 4-chloromethylstyrene: 3-chloromethylstyrene-95:5 (molar ratio), purity 96.87% by weight) was added dropwise over 1.5 hours, and the mixture was allowed to react at 65° C. for 4 hours. 150 parts of toluene was added, and the organic layer was washed five times with 100 parts of water. The obtained organic layer was concentrated to obtain 60.3 parts of compound (F9) represented by formula (f-2) above. The HP-LC chart of the obtained compound (F9) is shown in FIG. 17. The ¹H-NMR data (deuterated chloroform) of the obtained compound is shown in FIG. 18. A signal derived from the terminal hydrogen of the vinyl group (two hydrogens per vinyl group) was observed at 4.80-5.80 ppm in the ¹H-NMR chart, and a signal derived from the structure modified with benzhydrol was observed at 2.80-3.30 ppm. The integral value of the peak derived from the terminal hydrogen of the vinyl group was 4.00, and the integral value of the peak derived from each hydrogen of the methine group modified with benzhydrol was 2.78. The average mₐᵥₑ of the values m in the above formula (f-2) calculated from these values was 1.39.

### Example 10

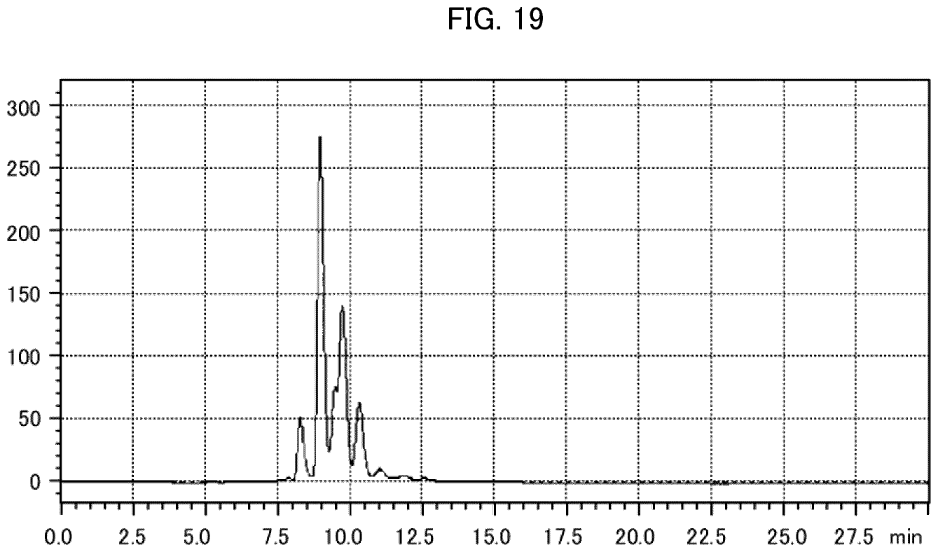

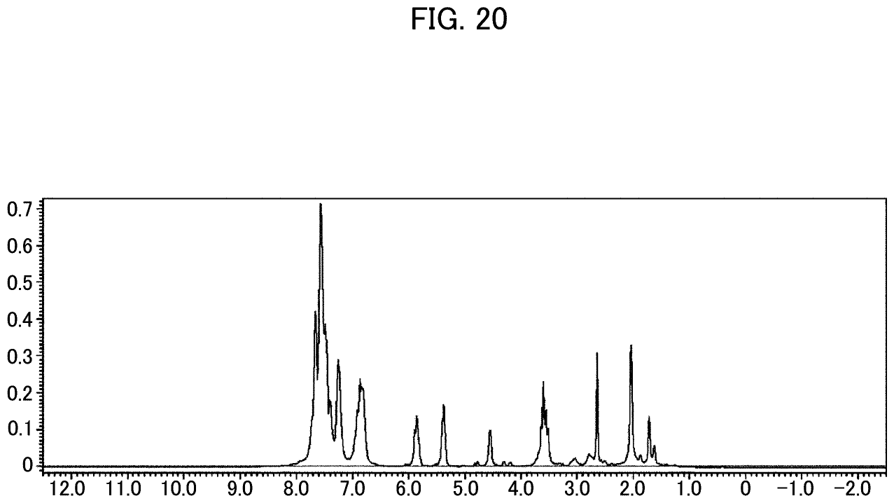

While purging with nitrogen into a flask equipped with a thermometer, a cooling tube, a stirrer, and a Dean-Stark tube, 16.6 parts of fluorene, 82 parts of methylcyclohexane, and 2.1 parts of methanesulfonic acid were added, and the temperature was raised to 95° C., and 12.2 parts of 1-phenylethyl alcohol were added dropwise over 1.5 hours while the internal temperature did not exceed 100° C., and the reaction was carried out for 4 hours while removing the generated water from the system using a Dean-Stark tube by azeotropic dehydration. The internal temperature was then cooled to 95° C., and 6.1 parts of 1-phenylethyl alcohol were added dropwise over 0.5 hours while the internal temperature did not exceed 100° C., and the reaction was carried out for 3 hours while removing the generated water from the system using a Dean-Stark tube by azeotropic dehydration. Thereafter, the internal temperature was cooled to 95° C., and 6.1 parts of 1-phenylethyl alcohol was added dropwise over 0.5 hours while the internal temperature did not exceed 100° C. The reaction was carried out for 7 hours while removing the generated water from the system using a Dean-Stark tube by azeotropic dehydration, and the internal temperature was cooled to 60° C.° C. 3.2 parts of a 30% aqueous sodium hydroxide solution was added, and methylcyclohexane was recovered until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (manufactured by AGC Seimi Chemical Co., Ltd., a mixture of 4-chloromethylstyrene and 3-chloromethylstyrene, 4-chloromethylstyrene: 3-chloromethylstyrene-95:5 (mol ratio), purity 96.87% by weight) was added dropwise over 2 hours, and the mixture was reacted at 65° C. for 3 hours. 150 parts of toluene was added, and the organic layer was washed five times with 100 parts of water. The obtained organic layer was concentrated to obtain 53 parts of compound (F10) represented by the following formula (f-3). The HP-LC chart of the obtained compound (F10) is shown in FIG. 19. In addition, the ¹H-NMR data (deuterated chloroform) of the obtained compound is shown in FIG. 20. A signal derived from the terminal hydrogen of the vinyl group (two hydrogens per vinyl group) was observed at 5.30-6.00 ppm in the 1H-NMR chart, and a signal derived from the structure modified with 1-phenylethyl alcohol was observed at 1.20-2.30 ppm. The integral value of the peak derived from the terminal hydrogen of the vinyl group was 4.00, and the integral value of the peak derived from each hydrogen of the methyl group modified with 1-phenylethyl alcohol was 5.60. The average mₐᵥₑ of the values m in the following formula (f-3) calculated from this was 0.93.

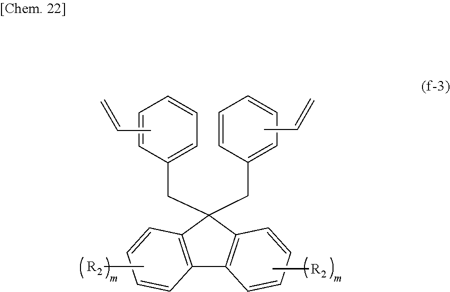

In the above formula (f-3), a plurality of R₂ each independently represent a hydrocarbon group represented by the following formula (f-a-3).

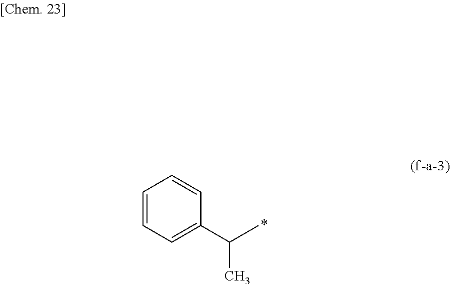

In the above formula (f-a-3), * represents the bonding position to the fluorene structure of formula (f-3).

### Example 11

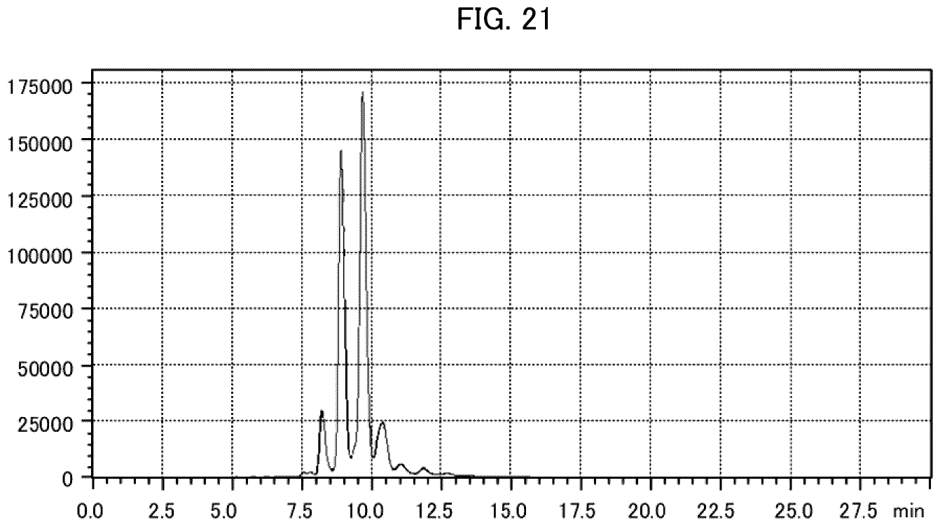

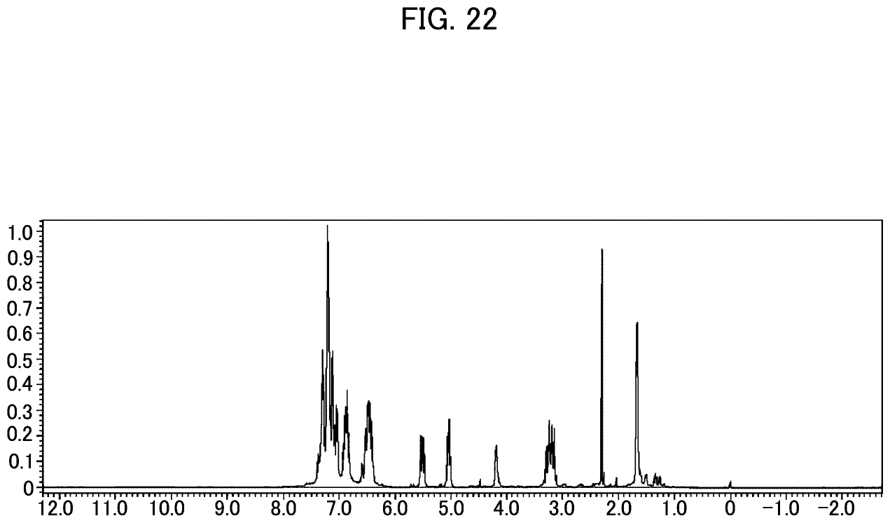

A flask equipped with a thermometer, a cooling tube, a stirrer, and a Dean-Stark tube was purged with nitrogen, and 16.6 parts of fluorene, 9.6 parts of methylcyclohexane, and 2.9 parts of methanesulfonic acid were added, and the temperature was raised to 125° C., and 12.2 parts of 1-phenylethyl alcohol were added dropwise over 1 hour while the internal temperature did not exceed 130° C., and the reaction was carried out for 3 hours at 130° C. while removing the generated water from the system using a Dean-Stark tube by azeotropic dehydration. The internal temperature was cooled to 125° C., and 6.1 parts of 1-phenylethyl alcohol were added dropwise over 30 minutes while the internal temperature did not exceed 130° C., and the reaction was carried out for 1 hour at 130° C. while removing the generated water from the system using a Dean-Stark tube by azeotropic dehydration. Subsequently, the internal temperature was cooled to 125° C., and 3.1 parts of 1-phenylethyl alcohol was dropped over 15 minutes while the internal temperature did not exceed 130° C., and the reaction was carried out at 130° C. for 3 hours while removing the generated water from the system using a Dean-Stark tube by azeotropic dehydration. The internal temperature was cooled to 80° C., 1.3 parts of sodium hydroxide was added, and methylcyclohexane was recovered until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (manufactured by AGC Seimi Chemical Co., Ltd., a mixture of 4-chloromethylstyrene and 3-chloromethylstyrene, 4-chloromethylstyrene: 3-chloromethylstyrene=95:5 (mol ratio), purity 96.87 wt %) was dropped over 1 hour, and the reaction was carried out at 65° C. for 3 hours. 150 parts of toluene was added, and the organic layer was washed five times with 100 parts of water. The obtained organic layer was concentrated to obtain 51.1 parts of compound (F11) represented by the above formula (f-3). The HP-LC chart of the obtained compound (F11) is shown in FIG. 21. In addition, the ¹H-NMR data (deuterated chloroform) of the obtained compound is shown in FIG. 22. A signal derived from the terminal hydrogen of the vinyl group (two hydrogens per vinyl group) was observed at 5.00-5.60 ppm in the ¹H-NMR chart, and a signal derived from the structure modified with 1-phenylethyl alcohol was observed at 1.00-1.90 ppm. The integral value of the peak derived from the terminal hydrogen of the vinyl group was 5.00, and the integral value of the peak derived from each hydrogen of the methyl group modified with 1-phenylethyl alcohol was 5.44. The average mₐᵥₑ of the values m in the above formula (f-3) calculated from this was 0.68.

### Example 12

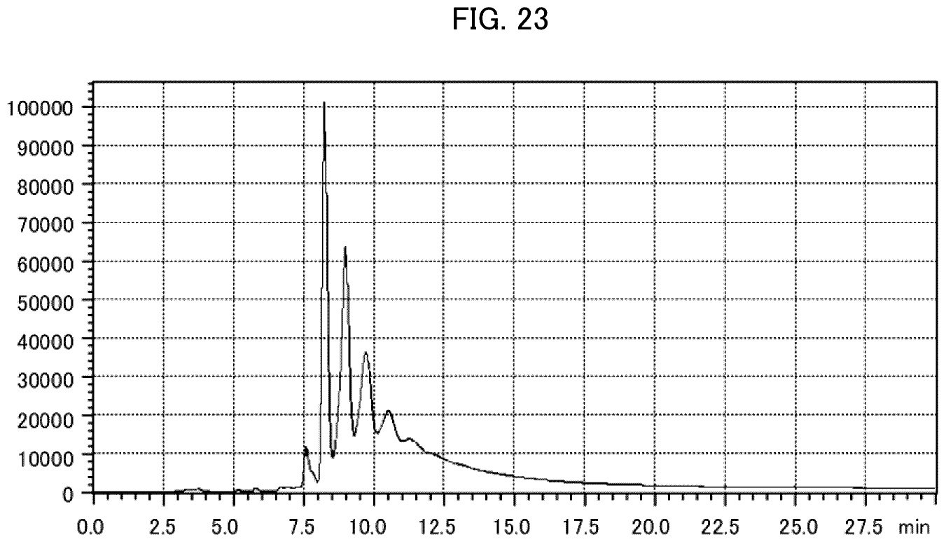

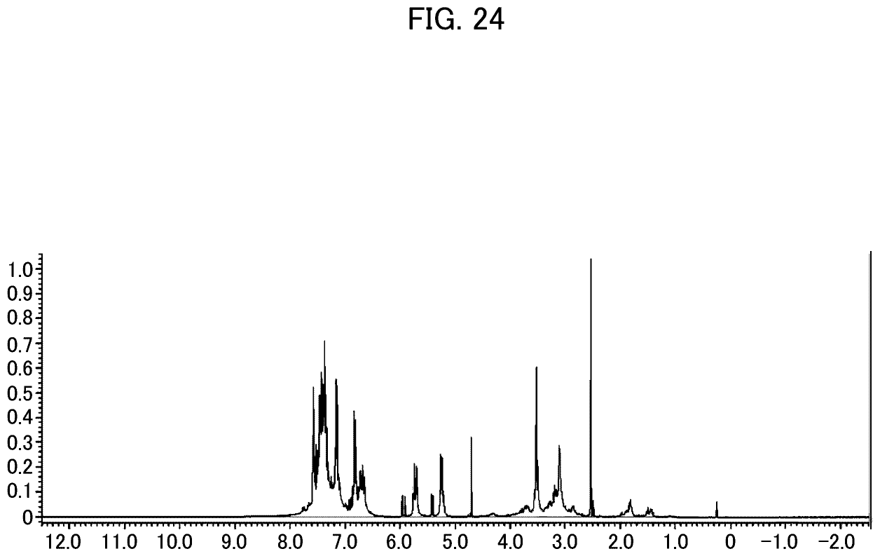

While purging with nitrogen into a flask equipped with a thermometer, a cooling tube, a stirrer, and a Dean-Stark tube, 16.6 parts of fluorene, 57.4 parts of methylcyclohexane, 24.4 parts of 2-phenylethyl alcohol, and 4.1 parts of methanesulfonic acid were added, and the temperature was raised to 180° C. The reaction was carried out for 28 hours while removing the generated water from the system by azeotropic dehydration using a Dean-Stark tube. The internal temperature was then cooled to 80° C., and 2.0 parts of sodium hydroxide were added. Then, 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 1.5 hours, and the mixture was allowed to react at 65° C. for 2 hours. 300 parts of toluene was added, and the organic layer was washed five times with 100 parts of water. The obtained organic layer was concentrated to obtain 22.8 parts of compound (F12) represented by the following formula (f-4). The HP-LC chart of the obtained compound (F12) is shown in FIG. 23. The ¹H-NMR data (deuterated chloroform) of the obtained compound is shown in FIG. 24. A signal derived from the terminal hydrogen of the vinyl group (two hydrogens per vinyl group) was observed at 5.15-5.97 ppm in the ¹H-NMR chart, and a signal derived from the structure modified with 2-phenylethyl alcohol was observed at 2.68-4.06 ppm. The integral value of the peak derived from the terminal hydrogen of the vinyl group was 4.00, and the integral value of the peak derived from each hydrogen of the methylene site modified with 2-phenylethyl alcohol was 8.50. The average mₐᵥₑ of the values m in the following formula (f-4) calculated from these values was 1.06.

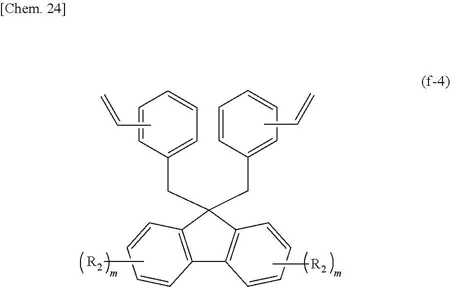

In the above formula (f-4), a plurality of R₂ each independently represent a hydrocarbon group represented by the following formula (f-a-4).

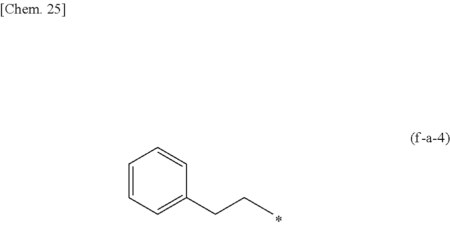

In the above formula (f-a-4), * represents the bonding position to the fluorene structure of formula (f-4).

### Example 13

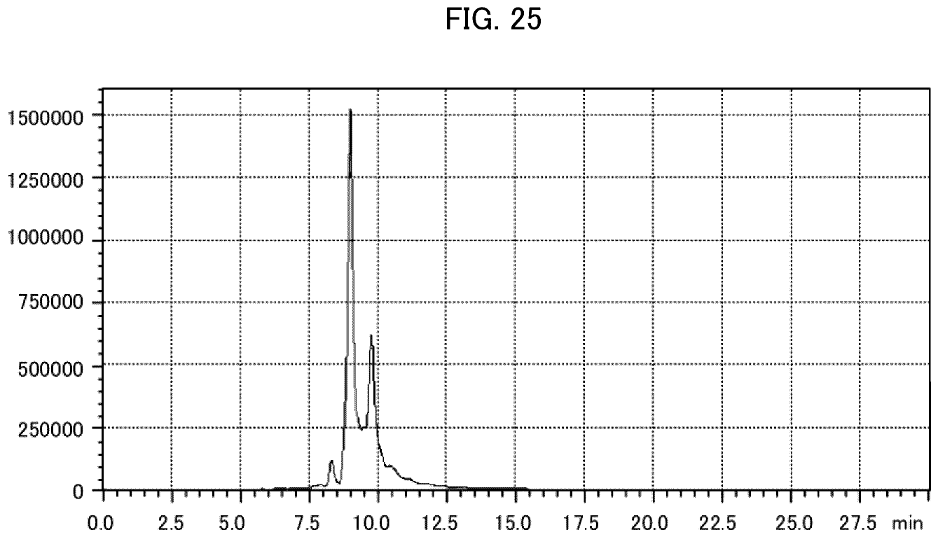

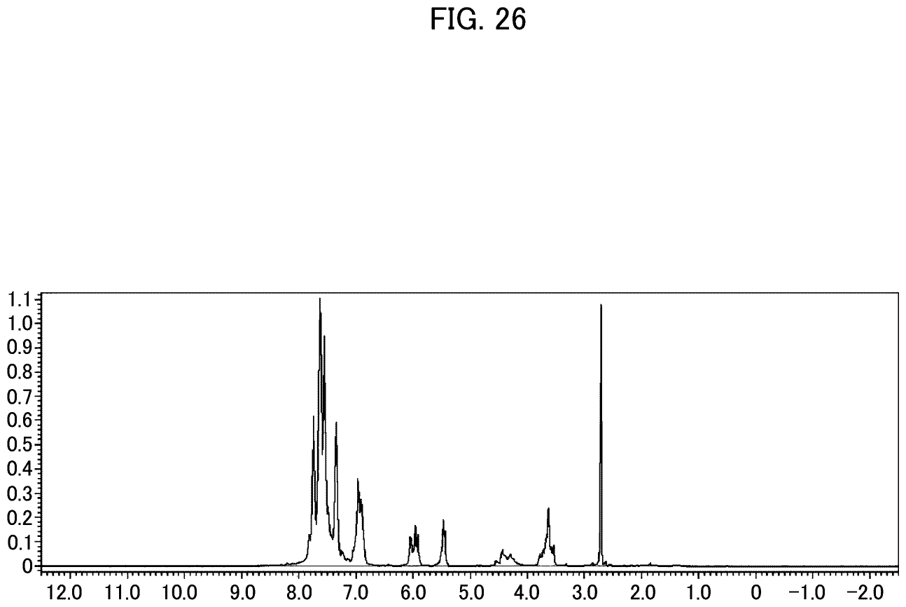

While nitrogen purging was applied to a flask equipped with a thermometer, a cooling tube, a stirrer, and a Dean-Stark tube, 16.6 parts of fluorene, 18.4 parts of benzhydrol, 70 parts of methylcyclohexane, and 1.8 parts of methanesulfonic acid were added, and the temperature was raised to 100° C., and the reaction was carried out for 6 hours while removing the generated water from the system using a Dean-Stark tube by azeotropic dehydration. After cooling to 70° C., 10.8 parts of benzyl alcohol was added, and the temperature was raised again to 100° C., and the reaction was carried out for 4 hours while removing the generated water from the system using a Dean-Stark tube by azeotropic dehydration. 2.6 g of 30% aqueous sodium hydroxide was added, and methylcyclohexane was collected until the internal temperature reached 140° C. The internal temperature was cooled to 80° C., and 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (AGC Seimi Chemical Co., Ltd., mixture of 4-chloromethylstyrene and 3-chloromethylstyrene, 4-chloromethylstyrene: 3-chloromethylstyrene=95:5 (molar ratio), purity 96.87% by weight) was added dropwise over 2 hours, and the mixture was allowed to react at 65° C. for 4 hours. 150 parts of toluene was added, and the organic layer was washed five times with 100 parts of water. The obtained organic layer was concentrated to obtain 60 parts of compound (F13) represented by the following formula (f-5). The HP-LC chart of the obtained compound (F13) is shown in FIG. 25. In addition, the ¹H-NMR data (deuterated chloroform) of the obtained compound is shown in FIG. 26. A signal derived from the terminal hydrogen of the vinyl group (two hydrogens per vinyl group) was observed at 5.40-6.10 ppm in the ¹H-NMR chart, and a signal derived from the structure modified with benzyl alcohol and benzhydrol was observed at 3.50-4.60 ppm. The integral value of the peak derived from the terminal hydrogen of the vinyl group was 4.00, and the integral value of the peak derived from each hydrogen of the benzyl group and methine group modified with benzyl alcohol and benzhydrol was 3.20. The average mₐᵥₑ of the values m in the following formula (f-5) calculated from this was 1.07. In addition, the value obtained by dividing the number of introduced substituents represented by the following formula (f-a-5) calculated from the amounts of benzhydrol and benzyl alcohol charged by the number of introduced substituents represented by the following formula (f-b-5) was 1.0.

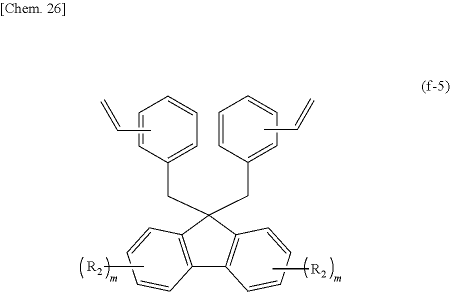

In the above formula (f-5), a plurality of R₂ each independently represent a hydrocarbon group represented by the following formula (f-a-5) or (f-b-5).

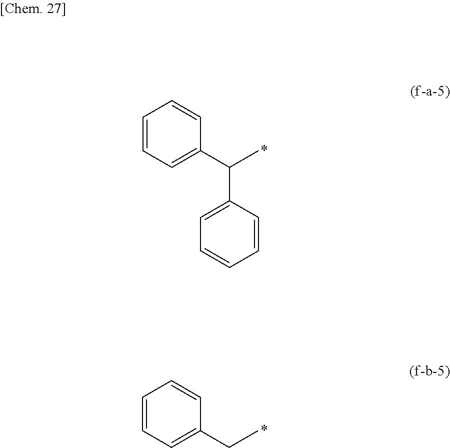

In the above formulas (f-a-5) and (f-b-5), * represents the bonding position to the fluorene structure of formula (f-5).

### Example 14

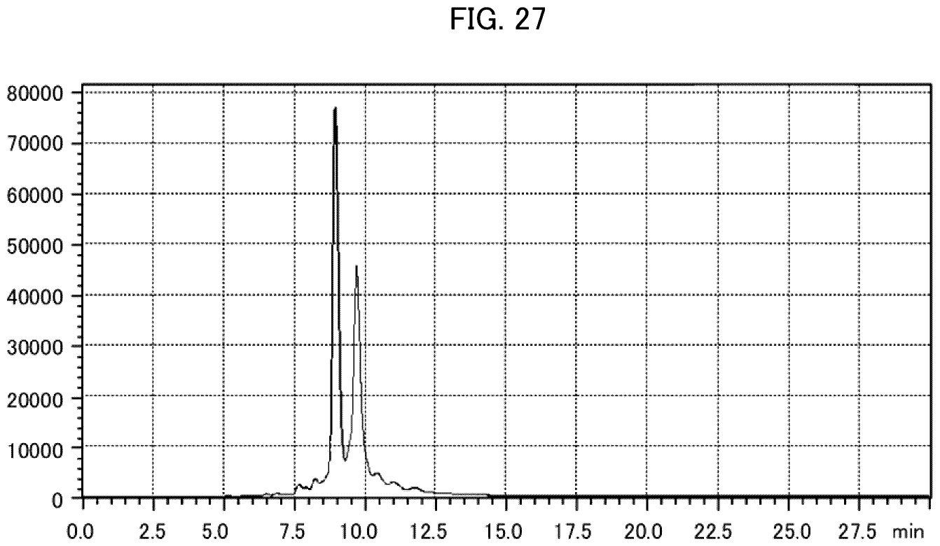

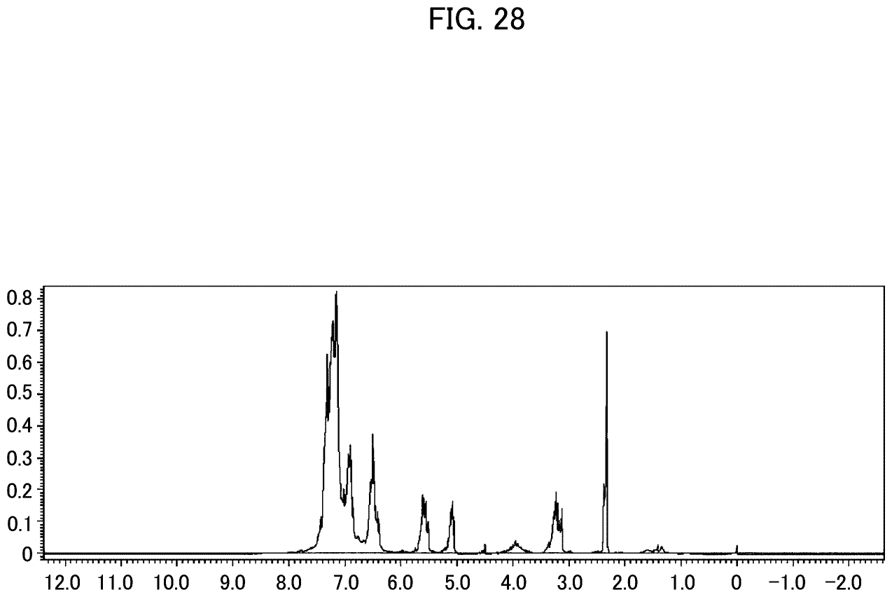

While purging with nitrogen into a flask equipped with a thermometer, a cooling tube, a stirrer, and a Dean-Stark tube, 16.6 parts of fluorene, 18.4 parts of benzhydrol, 70 parts of methylcyclohexane, and 2.2 parts of methanesulfone were added, and the temperature was raised to 98° C., and the reaction was carried out for 4 hours while removing the generated water from the system by azeotropic dehydration using a Dean-Stark tube. After cooling the internal temperature to 70° C., 9.2 parts of benzhydrol were added, and the reaction was carried out for 3 hours at 98° C. while removing the generated water from the system by azeotropic dehydration using a Dean-Stark tube. After cooling the internal temperature to 70° C., 5.4 parts of benzyl alcohol was added, and the temperature was raised again to 98° C., and the reaction was carried out for 3 hours while removing the generated water from the system by azeotropic dehydration using a Dean-Stark tube. 3.4 parts of a 30% aqueous sodium hydroxide solution was added, and methylcyclohexane was collected until the internal temperature reached 140° C. The internal temperature was cooled to 80° C., 100 parts of dimethyl sulfoxide and 12 parts of sodium hydroxide were added, and the mixture was stirred at 60° C. for 30 minutes. Thereafter, while maintaining the internal temperature at 65° C. or less, 29.0 parts of CMS-14 (manufactured by AGC Seimi Chemical Co., Ltd., a mixture of 4-chloromethylstyrene and 3-chloromethylstyrene, 4-chloromethylstyrene: 3-chloromethylstyrene=95:5 (mol ratio), purity 96.87% by weight) was added dropwise over 2 hours, and the mixture was reacted at 65° C. for 2 hours. 150 parts of toluene was added, and the organic layer was washed five times with 100 parts of water. The obtained organic layer was concentrated to obtain 63.4 parts of compound (F14) represented by the above formula (f-5). The HP-LC chart of the obtained compound (F14) is shown in FIG. 27. ¹H-NMR data (deuterated chloroform) of the obtained compound is shown in FIG. 28. A signal derived from the terminal hydrogen of the vinyl group (two hydrogens per vinyl group) was observed at 5.00-5.80 ppm in the ¹H-NMR chart, and a signal derived from the structure modified with benzyl alcohol and benzhydrol was observed at 3.10-4.30 ppm. The integral value of the peak derived from the terminal hydrogen of the vinyl group was 4.00, and the integral value of the peak derived from each hydrogen of the benzyl group and methine group modified with benzyl alcohol and benzhydrol was 4.21. The average mₐᵥₑ of the values m in the above formula (f-5) calculated from this was 0.84. The value obtained by dividing the number of introduced substituents represented by the above formula (f-a-5) calculated from the amount of benzhydrol and benzyl alcohol charged by the number of introduced substituents represented by the above formula (f-b-5) was 3.0.

### Comparative Synthesis Example 1

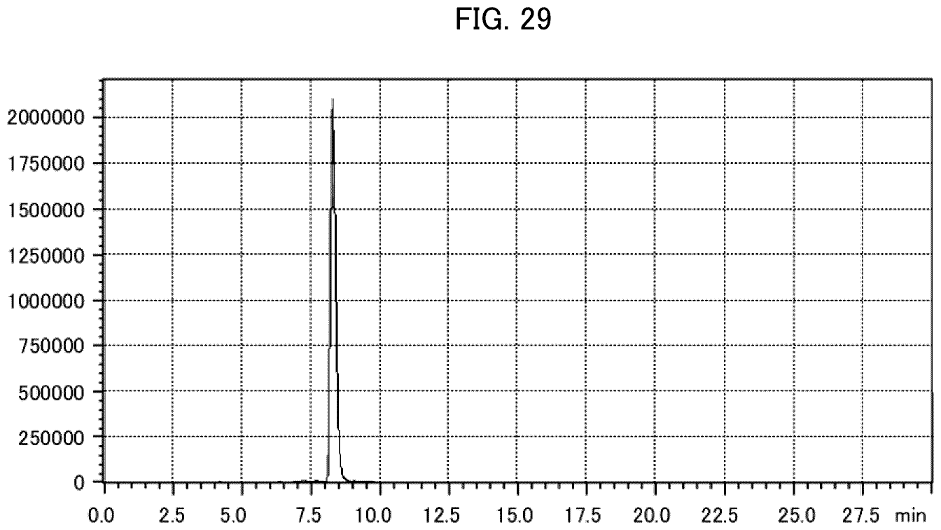

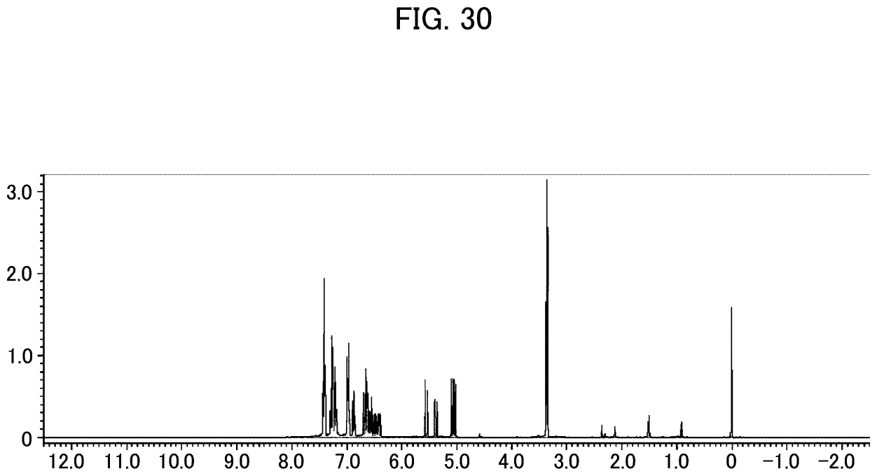

While purging with nitrogen into a flask equipped with a thermometer, a cooling tube, and a stirrer, 133 parts of methyl isobutyl ketone, 33.3 parts of fluorene, 1.9 parts of tetrabutylammonium bromide, 0.49 parts of hydroquinone, and 64 parts of 50 wt % aqueous sodium hydroxide solution were added, and the internal temperature was raised to 60° C. Then, 71.2 parts of CMS-P (manufactured by AGC Seimi Chemical Co., Ltd., a mixture of 4-chloromethylstyrene and 3-chloromethylstyrene, 4-chloromethylstyrene: 3-chloromethylstyrene=1:1 (mol ratio), purity 95.59 wt %) was dropped over 1 hour and reacted at 60° C. for 9 hours. Neutralized with 41.6 parts of 35 wt % aqueous hydrochloric acid solution, and the organic layer was washed three times with 100 parts of water. Recrystallized with toluene and methanol, 35.6 parts of compound (F15) represented by the following formula (f-6) was obtained. The HP-LC chart of the obtained compound (F15) is shown in FIG. 29, and the ¹H-NMR data (deuterated chloroform) is shown in FIG. 30.

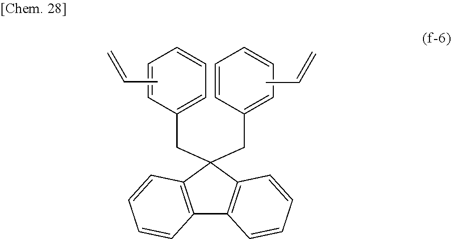

### Examples 15-27, Comparative Examples 1 and 2

The compounds (F1, F3-F15) obtained in Examples 1, 3-14, and Comparative Synthesis Example 1, and OPE-2St (polyphenylene ether compound, manufactured by Mitsubishi Gas Chemical Co., Ltd.) were used in the amounts shown in Table 1, and were sandwiched between mirror-finished copper foils (T4X: manufactured by Fukuda Metal Copper Foil Co., Ltd.) and vacuum-press molded, followed by curing at 220° C. for 2 hours. At this time, a piece of cushion paper with a thickness of 250 μm was used as a spacer, with the center cut out to 150 mm in length and width. For the evaluation, a test piece was cut out to the desired size using a laser cutter as necessary, and the evaluation was performed.

### <Dielectric Constant Test/Dielectric Loss Tangent Test>

Using a 10 GHz cavity resonator manufactured by AET Co., Ltd., tests were performed by a cavity resonator perturbation method at 25° C. The sample size was 1.7 mm wide×100 mm long, and the thickness was 0.1 mm. The evaluation results are shown in Table 1.

TABLE 1

| | Example | Example | Example | Example | Example | Example | Example | Example |
|---|---|---|---|---|---|---|---|---|
| | 15 | 16 | 17 | 18 | 19 | 20 | 21 | 22 |
| F1 | 100 | | | | | | | |
| F3 | | 100 | | | | | | |
| F4 | | | 100 | | | | | |
| F5 | | | | 100 | | | | |
| F6 | | | | | 100 | | | |
| F7 | | | | | | 100 | | |
| F8 | | | | | | | 100 | |
| F9 | | | | | | | | 100 |
| F10 | | | | | | | | |
| F11 | | | | | | | | |
| F12 | | | | | | | | |
| F13 | | | | | | | | |
| F14 | | | | | | | | |
| F15 | | | | | | | | |
| OPE-2St | | | | | | | | |
| Dielectric Constant | 2.41 | 2.36 | 2.51 | 2.63 | 2.41 | 2.51 | 2.52 | 2.50 |
| Dielectric Loss Tangent | 0.00081 | 0.00120 | 0.00114 | 0.00098 | 0.00062 | 0.00136 | 0.00165 | 0.00172 |

| | Example | Example | Example | Example | Example | Comparative | Comparative |
|---|---|---|---|---|---|---|---|
| | 23 | 24 | 25 | 26 | 27 | Example 1 | Example 2 |
| F1 | | | | | | | |
| F3 | | | | | | | |
| F4 | | | | | | | |
| F5 | | | | | | | |
| F6 | | | | | | | |
| F7 | | | | | | | |
| F8 | | | | | | | |
| F9 | | | | | | | |
| F10 | 100 | | | | | | |
| F11 | | 100 | | | | | |
| F12 | | | 100 | | | | |
| F13 | | | | 100 | | | |
| F14 | | | | | 100 | | |
| F15 | | | | | | 100 | |
| OPE-2St | | | | | | | 100 |
| Dielectric Constant | 2.41 | 2.42 | 2.59 | 2.50 | 2.58 | 2.51 | 2.51 |
| Dielectric Loss Tangent | 0.00080 | 0.00117 | 0.00190 | 0.00087 | 0.00176 | 0.00410 | 0.00395 |

From the results in Table 1, it was confirmed that the compounds of the present invention have a low dielectric tangent and excellent dielectric properties.

### Examples 28-35

Each compound (F1, F3-F5, F7, F10, F12, F13) obtained in Examples 1, 3-5, 7, 10, 12, and 13 was used in the amount shown in Table 2, sandwiched between mirror-finished copper foil (T4X: manufactured by Fukuda Metal Copper Foil Co., Ltd.), vacuum press molded, and cured at 220° C. for 2 hours. In this case, a cushion paper with a center cutout of 150 mm length and width was used as a spacer. For evaluation, a test piece was cut out to a desired size using a laser cutter as necessary, and evaluation was performed. The thickness of the test piece was adjusted depending on the thickness of the cushion paper.

### <Glass Transition Temperature (Tg)>

Dynamic viscoelasticity measuring device: TA-instruments, DMA-2980

Measurement temperature range: −30 to 280° C.

Heating rate: 2° C./min

Frequency: 10 Hz

Test piece size: A cutout to 5 mm×50 mm was used (thickness was 0.1 mm).

Tg: The peak point of tan 8 (=loss modulus/storage modulus) was taken as the glass transition temperature (Tg).

TABLE 2

| | Example | Example | Example | Example | Example | Example | Example | Example |
|---|---|---|---|---|---|---|---|---|
| | 28 | 29 | 30 | 31 | 32 | 33 | 34 | 35 |
| F1 | 100 | | | | | | | |
| F3 | | 100 | | | | | | |
| F4 | | | 100 | | | | | |
| F5 | | | | 100 | | | | |
| F7 | | | | | 100 | | | |
| F10 | | | | | | 100 | | |
| F12 | | | | | | | 100 | |
| F13 | | | | | | | | 100 |
| Tg(° C.) | 161 | 201 | 207 | 191 | 231 | 201 | 164 | 204 |

This application claims priority based on Japanese Patent Application No. 2023-046177 filed on Mar. 23, 2023, Japanese Patent Application No. 2023-137647 filed on Aug. 28, 2023, Japanese Patent Application No. 2023-137648 filed on Aug. 28, 2023, Japanese Patent Application No. 2023-204993 filed on Dec. 5, 2023, and Japanese Patent Application No. 2023-214747 filed on Dec. 20, 2023.

### INDUSTRIAL APPLICABILITY

The compound of the present invention is suitably used for electric and electronic parts such as semiconductor encapsulants, printed wiring boards, and build-up laminates.

1. A compound represented by the following formula (1): 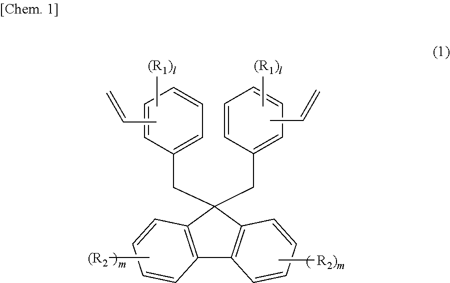
in the above formula (1),
each of the multiple R₁ independently represents a hydrogen atom or a hydrocarbon group having 1 to 5 carbon atoms;
each of the multiple R₂ independently represents a hydrocarbon group represented by the following formula (a),
each of the multiple 1 independently represents an integer of 1 to 4,
each of the multiple m independently represents an integer of 0 to 4, and the average mₐᵥₑ of the values m is 0<mₐᵥₑ≤4, 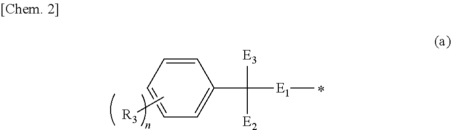
in the above formula (a),
* represents a bonding position to the fluorene structure of formula (1),
each of the multiple R₃ independently represents a hydrogen atom or a hydrocarbon group having 1 to 5 carbon atoms,
E₁ represents a direct bond or a methylene group,
E₂ and E₃ represent a hydrogen atom, a hydrocarbon group having 1 to 5 carbon atoms, or
a substituted or unsubstituted phenyl group, and
n represents an integer of 1 to 5.

2. The compound according to claim 1, wherein the hydrocarbon group represented by formula (a) is one or more selected from the following formulas (a-1) to (a-6): 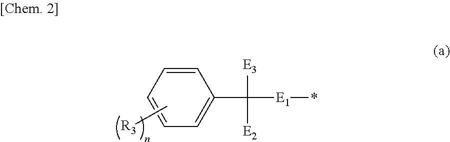
in the above formulas (a-1) to (a-6), * represents a bonding position to the fluorene structure of formula (1).

3. A compound obtained by reacting a compound represented by the following formula (A) with a compound represented by the following formula (B): 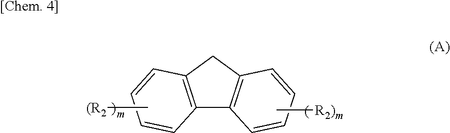
in the above formula (A),
each of the multiple R₂ independently represents a hydrocarbon group represented by the following formula (a),
each of the multiple m independently represents an integer of 0 to 4, and the average mₐᵥₑ of the values m is 0<mₐᵥₑ≤4, 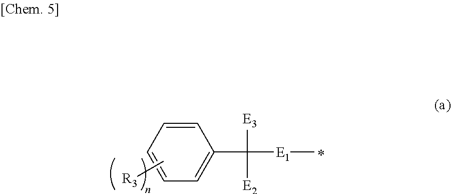
in the above formula (a),
* represents a bonding position to the fluorene structure of formula (1),
each of the multiple R₃ independently represents a hydrogen atom or a hydrocarbon group having 1 to 5 carbon atoms,
E₁ represents a direct bond or a methylene group,
E₂ and E₃ represent a hydrogen atom, a hydrocarbon group having 1 to 5 carbon atoms, or
a substituted or unsubstituted phenyl group, n represents an integer of 1 to 5, 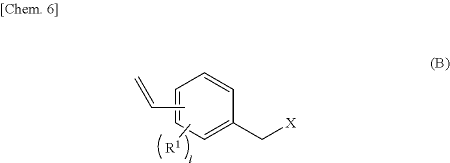
in the above formula (B), each of the multiple R₁ independently represents a hydrogen atom or a hydrocarbon group having 1 to 5 carbon atoms,
l represents an integer of 1 to 4, and
X represents a halogen atom.

4. A curable resin composition comprising the compound according to claim 1.

5. A cured product obtained by curing the compound according to claim 1.

6. A cured product obtained by curing the curable resin composition according to claim 4.

7. A method for producing a compound obtained by reacting a compound represented by the following formula (A) with a compound represented by the following formula (B) in an aprotic polar solvent in the presence of a basic catalyst: 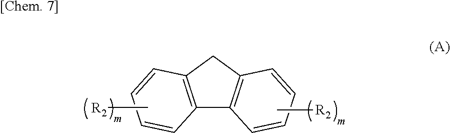
in the above formula (A),
the multiple R₂ each independently represent a hydrocarbon group represented by the following formula (a),
the multiple m's each independently represent an integer of 0 to 4, and
the average mₐᵥₑ of the values m is 0<mₐᵥₑ≤4, 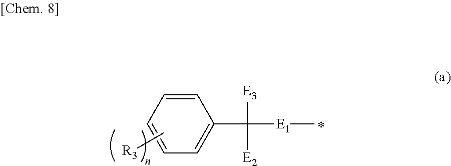
in the above formula (a),
* represents a bonding position to the fluorene structure of formula (1),
the multiple R₃ each independently represent a hydrogen atom or a hydrocarbon group having 1 to 5 carbon atoms,
E₁ represents a direct bond or a methylene group,
E₂ and E₃ each represent a hydrogen atom, a hydrocarbon group having 1 to 5 carbon atoms, or a substituted or unsubstituted phenyl group,
n represents an integer of 1 to 5, 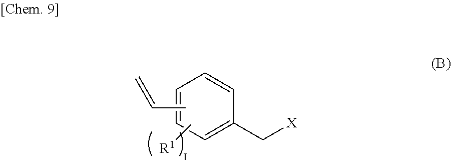
in the above formula (B), each of the multiple R₁ independently represents a hydrogen atom or a hydrocarbon group having 1 to 5 carbon atoms,
l represents an integer of 1 to 4, and
X represents a halogen atom.

8. A curable resin composition comprising the compound according to claim 2.

9. A cured product obtained by curing the curable resin composition according to claim 8.

10. A cured product obtained by curing the compound according to claim 2.

11. A curable resin composition comprising the compound according to claim 3.

12. A cured product obtained by curing the curable resin composition according to claim 11.

13. A cured product obtained by curing the compound according to claim 3.
